SEQUENCE LISTING (37 sequences)

SEQ ID NO: 1 - DNA, 2496 bp, Arabidopsis thaliana
  LIGHT5 TZP coding sequence
atgggagatggagatgagcaaagtaaagagttaggaggagtttcgtcttcgagtcgtcgttgttcttccggtactgctggtgctgcaaatgcagaagcaagaatgaagtttgctgctgttgatgctataacagaattagtgtggtctcctagcaacggtctaagcttaagatgtgcagatatcagtttcacgggaaaggcaaaattactctctcctaacttctttgatataggactaactaacatggcgattcactcaaatagcacaagtattgaagaccaagaagatcacgtagatgtcgaattgagaaaccgagatcaagtgaaccaagcaatgattggtggttctgttgaggatatgaaacctgaaatggttgaggataaggttgaaaccaatgatgatataaagaatgaagaagcaggatgttccaaaagatcttcagatagtccgaaagctatggaaggagaaaccagagacttgttggttaatgaacagttgaggatggaaagtgctggatctcaagaagaaggggataaggcgcataatagagttgatcgtttagaatcaatggatgagaacaacctcgcaactcttgctgttgtagcatgtgaaggtaaaggggactatttgccggagggtgaggctggacctagtggttcctacaggcgtcgtgagaaagcaaaagggaaagagaaagctttatctgatgaaaactttggtggtgatggtgaagatgaggatgaggagagttttggtagtgtagaaagttgcaatagtgcgggtttgctctcaaggggaaagaaaagaccaggctttgaggagcagctgatttttggaagcaaacggctcaaaacgttaaatcaagaatgtttggggtcaacttcaaagctcaagcaagatagttccttcatgaattggatatcaaatatgactaaaggaatctggaaaggtaatgaagaagacaattctccttttgtagctcttaccaccacctcaaatgctaatggtcatggccaagtcaatgccatcgttgaccaacaacaattgagtccatgctgtgtgaaggaaaatagtggatgcagaaacaccggtttccagtcttttttccagtctatatattgtccgaagaaacaaagtcaagatgtcgtcgacatggatttcccgaatgatgttaatgctgctcctttgcaggaacttccctggattcctgaacattgcgacatttccaaaggggatgacttgtcttcatctggtaatgagattggtccagtggctgaacccaacatttcatcaggaaaagttgtatttaatcagacaagcaaaacacagtcttcagagaacaaacgtgaggacaaagagccaaacatctcgttgatgtctcttagtaagtctaagccaaatgaagagccaaaaacatgcggtgaagctgatggaaaggttagtccatgtttaaccaacagaaactctggtcttaaaagtttgtggattagtcggttttcttctaaaggttcgttccctcagaaaaaggcaagtgaaacggctaaagaggccaatgcttcagcctcagacgcagctaaaacccgtgattcacggaaaatgctggcagacaagaatgttataagacccagtatcagctcggtagatgggcccgataaaccggacactgtccttcctatagtttcgtcaatgagaatagagtcttcagaagcaatggcttctttgtttgctagaagattagaagcaatgaaaagcatcatgccctcaggttctttagctgagaatgcagaggaggaacagagggatctaatttgtttctactgcggtaaaaagggtcattgtttacgggattgtttggaagtaactgatactgagctcagagatctagtacagaacataagtgtgcggaatggaagagaagaagcgtcaagtctatgcattagatgttttcagcttagtcactgggctgcaacatgcccgaatgctcccctgtatggttcaggagctgaaggtagagctatgaagaacgctttagcttctacctctggcatgaagctgcccataagtggtttcacagacgtaccaagggcggtctttgatgctgttcaagtgcttcgcctgagccgcacagacgttctcaaatggataaacaccaagaagtctgtatcgggtctcgaagggttcttcttgcggttaaggctcgggaaatgggaagaagggcttggaggaacagggtattatgtagctcgcatagatggggacacagagggacaaagctcaaggagacattcagagaaaagcttaatttcagttaaggttaaaggtgtgacttgccttgttgagtctcagtttatctcaaaccaagactttcttgaggaggagttaaaggcctggtggcagagcgcggggaaaagtgccagaacaagcggctacgacggcatcccgtcggcggaggaacttagtcggaaaattcagcagagaaaaatgttaggcttttag

SEQ ID NO: 2 - protein, 831 aa, Arabidopsis thaliana
  LIGHT5 TZP protein
MGDGDEQSKELGGVSSSSRRCSSGTAGAANAEARMKFAAVDAITELVWSPSNGLSLRCADISFTGKAKLLSPNFFDIGLTNMAIHSNSTSIEDQEDHVDVELRNRDQVNQAMIGGSVEDMKPEMVEDKVETNDDIKNEEAGCSKRSSDSPKAMEGETRDLLVNEQLRMESAGSQEEGDKAHNRVDRLESMDENNLATLAVVACEGKGDYLPEGEAGPSGSYRRREKAKGKEKALSDENFGGDGEDEDEESFGSVESCNSAGLLSRGKKRPGFEEQLIFGSKRLKTLNQECLGSTSKLKQDSSFMNWISNMTKGIWKGNEEDNSPFVALTTTSNANGHGQVNAIVDQQQLSPCCVKENSGCRNTGFQSFFQSIYCPKKQSQDVVDMDFPNDVNAAPLQELPWIPEHCDISKGDDLSSSGNEIGPVAEPNISSGKVVFNQTSKTQSSENKREDKEPNISLMSLSKSKPNEEPKTCGEADGKVSPCLTNRNSGLKSLWISRFSSKGSFPQKKASETAKEANASASDAAKTRDSRKMLADKNVIRPSISSVDGPDKPDTVLPIVSSMRIESSEAMASLFARRLEAMKSIMPSGSLAENAEEEQRDLICFYCGKKGHCLRDCLEVTDTELRDLVQNISVRNGREEASSLCIRCFQLSHWAATCPNAPLYGSGAEGRAMKNALASTSGMKLPISGFTDVPRAVFDAVQVLRLSRTDVLKWINTKKSVSGLEGFFLRLRLGKWEEGLGGTGYYVARIDGDTEGQSSRRHSEKSLISVKVKGVTCLVESQFISNQDFLEEELKAWWQSAGKSARTSGYDGIPSAEELSRKIQQRKMLGF

SEQ ID NO: 3 - DNA, 3766 bp, Arabidopsis thaliana
  LIGHT5 TZP genomic sequence
ggcgctgtgtcgtcttcgtcttcttcttccttcttcttcttcatctctctctttcgttttctcagtaagtgaaggatttgcagaagtagatgattgaatatccatggaggtaatttatggtttctaatttggttttaattctttgaaagtttgatttttttatgttatgagatttctgggtttttgattctcaagtttttatggattttagggttttcagtaattgcaaaactttattctgaagattgagtgattgcttgagatataggttatcaatttcctttatccctaatattccaaatctcgaaatttcaaattacttctctaagttgttctatgagtttatgatcttagaaaataaatctagtgttttgattactctgttttgcatatttttgatgggtttgtgttacttgttttgtaagctaagtgatattcatttttgtggttgaggtcaatagtgtatggattctggttctttatgtctcagggtgattggtttcaggaggtatgggagatggagatgagcaaagtaaagagttaggaggagtttcgtcttcgagtcgtcgttgttcttccggtactgctggtgctgcaaatgcagaagcaagaatgaagtttgctgctgttgatgctataacagaattagtgtggtctcctagcaacggtctaagcttaagatgtgcagatatcagtttcacgggaaaggcaaaattactctctcctaacttctttgatataggactaactaacatggcgattcactcaaatagcacaagtattgaagaccaagaagatcacgtagatgtcgaattgagaaaccgagatcaagtgaaccaagcaagtaatgaaatgaatagaccattttcaggttgctatatgataagttatcttttttgataatgagagttcttgtgtgtttgcagtgattggtggttctgttgaggatatgaaacctgaaatggttgaggataaggttgaaaccaatgatgatataaagaatgaagaagcaggatgttccaaaagatcttcaggtaaaatggttatatcaagttaataatgtttgcgtctcttccatttatgacaactatggattttgagttggtttcttcgtcttttgtgcttgttttggttatcgagtagatagtccgaaagctatggaaggagaaaccagagacttgttggttaatgaacagttgaggatggaaagtgctggatctcaagaagaaggggataaggcgcataatagagttgatcgtttagaatcaatggatgagaacaacctcgcaactcttgctgttgtagcatgtgaaggtaaaggggactatttgccggagggtgaggctggacctagtggttcctacaggcgtcgtgagaaagcaaaagggaaagagaaagctttatctgatgaaaactttggtggtgatggtgaagatgaggatgaggagagttttggtagtgtagaaagttgcaatagtgcgggtttgctctcaaggggaaagaaaagaccaggctttgaggagcagctgatttttggaagcaaacggctcaaaacgttaaatcaagaatgtttggggtcaacttcaaagctcaagcaagatagttccttcatgaattggatatcaaatatgactaaaggaatctggaaaggtaatgaagaagacaattctccttttgtagctcttaccaccacctcaaatgctaatggtcatggccaagtcaatgccatcgttgaccaacaacaattgagtccatgctgtgtgaaggaaaatagtggatgcagaaacaccggtttccagtcttttttccagtctatatattgtccgaagaaacaaagtcaagatgtcgtcgacatggatttcccgaatgatgttaatgctgctcctttgcaggaacttccctggattcctgaacattgcgacatttccaaaggggatgacttgtcttcatctggtaatgagattggtccagtggctgaacccaacatttcatcaggaaaagttgtatttaatcagacaagcaaaacacagtcttcagagaacaaacgtgaggacaaagagccaaacatctcgttgatgtctcttagtaagtctaagccaaatgaagagccaaaaacatgcggtgaagctgatggaaaggttagtccatgtttaaccaacagaaactctggtcttaaaagtttgtggattagtcggttttcttctaaaggttcgttccctcagaaaaaggcaagtgaaacggctaaagaggccaatgcttcagcctcagacgcagctaaaacccgtgattcacggaaaatgctggcagacaagaatgttataagacccagtatcagctcggtagatgggcccgataaaccggacactgtccttcctatagtttcgtcaatgagaatagagtcttcagaagcaatggcttctttgtttgctagaagattagaagcaatgaaaagcatcatgccctcaggttctttagctgagaatgcagaggaggaacagagggatctaatttgtttctactgcggtaaaaagggtcattgtttacgggattgtttggaagtaactgatactgagctcagagatctagtacagaacataagtgtgcggaatggaagagaagaagcgtcaagtctatgcattagatgttttcagcttagtcactgggctgcaacatgcccgaatgctcccctgtatggttcaggagctgaaggtagagctatgaagaacgctttagcttctacctctggcatgaagctgcccataagtggtttcacagacgtaccaagggcggtctttgatgctgttcaagtgcttcgcctgagccgcacagacgttctcaagtaagatctaattcacacctaaaagactgtttcccttgtcacattatgaggctttaacatcttttgacatttcatttcagatggataaacaccaagaagtctgtatcgggtctcgaagggttcttcttgcggttaaggctcgggaaatgggaagaagggcttggaggaacagggtattatgtagctcgcatagatggttggtacactttcatataaatcaatactcaaactcgcgtatttctcgaatatttgcaactgaaatatgtttgttttaaccaggggacacagagggacaaagctcaaggagacattcagagaaaagcttaatttcagttaaggttaaaggtgtgacttgccttgttgagtctcagtttatctcaaaccaagactttcttgaggtatataataaaatttgaccaattatgtcaaaagttttgaaatttgctaatttgctaagttttcatctgaatattttgcattaatttatctgagcaggaggagttaaaggcctggtggcagagcgcggggaaaagtgccagaacaagcggctacgacggcatcccgtcggcggaggaacttagtcggaaaattcagcagagaaaaatgttaggcttttaggacatttgttcaagtagataaatgtttttaagttataggttttacgattacccgtgtattttttcggttgaaaaggagagtgtaaatttcttagttcagagggcatcgaatgtctcaaaccatacaacaatctaaaactggatgtccaatctatttagatttgcgagttttattcggatctttatgtctcgatgactatttattgtgattgttaagcttgtatagtctatgtgtgaaggatcaaagtatcatggaagaacaaagtacattgatatcaagagagatattgtggtgtcttgctcatttttc

SEQ ID NO: 4 - protein, 689 aa, Glycine max
  soybean GmTZPa
MMLPCDSRIHMAINKGKEKSLSDGDANVILSREENDSHSSVESCNSAGFFSTGKKRRNFQQQLIIGSKRVKKQIEESSGFKSYVKQDSSFMNWISNMVKGLQQSIQNDSNTLALTLTNPDHHNLLPDEKLFTCNMNQDPEPKNTGFKSFFQSIYCPSLKNGGTRMSHQEGKSSDDLEPGNMEHGIDATPITYCAENNSLSKLRLQSNKFEVSIGGNDAGPSSQPKVKPLNFFNCQESSKNNPVETKNYSILGHSKDKEEVASHSSSTKQNTDDNDNIDSNALPDRKEEENICHRRDNLGSLWITRFSPKFTAPLREQPANDTEASTDLKEDKGNNDHKSMYMFKPLSSSPGLRNLEPMASMFARRFSAIKHIIPTNATDTTTQVNMLCLFCGTKGHQLSDCSAIAENKLEDLQKNIDSYGGLEEHSCLCIKCFQPNHWAISCPTSISTRKHELKANALVNDCGKQKHLIPSNEESARLLTDEDDRVLSGGSINDETDQRTGQNINLKLKSNEIITHKVGCNASFQKYCGSSLEENKFRENPISSPSKLTERQISHVPKKIFDAVKKLQLSRTDILKCINTHGSISQLDGFFLRLRLGKWEEGLGGTGYHVAYINETQSQRQCPEQNTRKCLSVKVGSIKCMVESQYISNHDFLEEEITEWWSNTSEAGAEIPSEEYLIEKFKKKEMLGL

SEQ ID NO: 5 - protein, 696 aa, Glycine max
  soybean GmTZPb
MLPCDKILPVLHSPCHSRIHMAINKGKEKSLSDGDANVMLSREENDSHSSVESCNSTGFFSTGKKRRNFQQQLIIGSKRVKKQIEESSGPKSYVKQDNSFMNWISNMVKGLSPSIQNDSNTLALTLANPDHHNLQPDEKLIACNMNQDPEPKNTGFKSIFQSICCPSLKNVGTRMSHQEGKSSQDLVPGNMEHGIDATPITCWAENNSLSKLCLQSNKFEVSTGGNDAGLSSQPKIKPLNFFNCHESSKNNPVETKNYSILGHSKDKEEVASHSSSTKQNTDNNDNIDSNVLCDRKEEENICHRRDNLGSLWITRFSPKFTAPLREQPANDTEVSTDLKEDKGNNDHKSMYLFKPLSSSPGFRNLEPTASMFGRRFGAIKQIIPTNATDTTTQVNMLCFFCGTRGHQLSDCLAIAENKLEDLQKNIDSYGGLEEHHCLCIKCFQPNHWAISCPTSISTRKHELKANALVNDCGKHLISSNEGSARLLTDEDDRVLSGGSINDETDQRAGQNINLKWKSNEIITHKVGCNASFKKYRGLSSEENKFRENPTLSPSKLAERQISQVPKEIFEAVKKLQLSRTDILKWINTRGSISQLDGFFLRLRLGKWEEGPGGTGYHVAYINETQSQRQCSEQNTRKSLSVKVGSIKCMVESQYISNHDFLEEEIMEWWSNTSQAGAEISSEEYIIEKFKKKEMLG

SEQ ID NO: 6 - protein, 783 aa, Oryza sativa
  rice OSTZP
MDVMNEGKETSDNFCVDKLEKEDEVGSCPTRYCNDTSHSLGSASRKEVMPIIAEKQAFCATTVHDERSWAANAWRARLVKAISQKDSVLPKNADNIHSTSAFGSIGNTENMPGKLTSMLGNRNDSSQDQAMQEKHKDGLIVARCESVSAVNPVARCELASGVNPLARHESTSGCNPRKLEKGKEKLIYDMSNCVSNTNEGDDSNESIESCPSTKAPKRKHGQFSAAEMTSGNKRCRREDNESSCSGLFHKNDSSFFNWMSTLTNGVKVFDETTAVPLNQKFSAATGEEFPTNPVPLQNNCGVPLQSVGFNSLFQSLYSQNVMITSRNTCHQSESSYTANRLTLGFKSSKPVSMGRETLNVATETLAAGRIQMDSYGDRGAFQNQMGIFPLRAERNQNGFHGSSSNAASGHKDDFSESLWVSRLLPKTPMKVMDTTRCDEETDFCSANPKGLGDSSSPQDFNVEKELNNSQYFTSKGSDNETTSSKCAAPQDENKPSETMASIFAKRLDALRHANTSAVHVAITCDHGTPKGRNHKTSSFVVSYNSHDEQESGQKTHKSSGGEGRIVLWTGDKGKEQLSPGNDKELGEKVLSKHENQNCEGSSDGKVVPPKCNLETNTYIEEIDRKRLQNKEGAPNSMENQPDNKQMVPYGIVPNDVYDEASVVFGALQRLRLSRSDIIRWMRSPVMHTTLDGFFLRLRFGKWEEALGGTGYHVARINGVLDKNRLSVTIRNSTCQVDSRFVSNHDFHQDELKAWWSAAMKSGWKLPSQEELNTKLRERELLRF

SEQ ID NO: 7 - protein, 970 aa, Populus trichocarpa
  poplar PtTZP
MDTNDKNIEPVIDLGFSLGYSNQCIQRRLKNDSGAGANAASSVDMTFVATNALSELVWSPKKGLSLKCADGTFSNQKPSLLRGAGPSDMVSGSNADKAIGKKVFMTPPEESDVRSEVAGRDNPTKFVTSDTGLFPLLSESMHKVKIGNYEFLAATDDHKEEMKTAVGLPFLQKMEDARNNKAEDIYDPINLQVDEISRTWETKFPSLSDETKLDVAQNGPTSKEPNVRIGGVGDASHTLQTEIVSASQVCSVEECESYDTNMQKAPLGREHFESPSCMEKERENNMGTGPYICPLEKLESTAENDFKTPHSENVCAVATEIVGSQNAKEVRSSSQQDDEILPKDNDCAIKQSPTYSRTRRYQMKGKAKALSDGNLNERMLDMDDDSHESVESCNSVGLFSTGKRQRNFDPHSYVGSKSIKTKIQESPGSSSFVKHDGSFMNWISNMMKGFLKSNEDEAPSLALTLANHKHGHEDRDKNLISCNRNQDQGCKTMGFHSLFQSLYCPKTKAQETVALNANTQTEGSKELGLDNKICDSNATPIPCRMVTDNVYKRFLQPNEKLNESTSGNGTASPALTKLLSTNIASGQEISGSNSAEKKNSCNMATDKEKNGTSSNSSRGKRKMNDAEQPSEGKATNTSGYRSDPLTSLWITRLSPKTSGPLSNRDLCHRRTGEALDGFTDFIRLKAQWQNHPSSYQDKNIVGAREEEHFTEDPVCMHNCANSTEVSFSINKVNGHHDEKSMCKMNSTLPFSRFRNSEAMASVFARRLDALMHIMPSYGTDDSSHGNLTCFFCGIKCHHVRDCPEIIDSELADILRNANSFNGANEFPCVCIRCFQSNHWAVACPSASSRTRHQAEYGASLVHESSPCKILLNPRNEDDAKQSDGKDSQLQAADAPTVRNGKLHEASASGKINMNMKPFERDTASSSGEKKLKENQVMPLSNFINSQIADVPKGIFDAVKRLRLSRTIILK

SEQ ID NO: 8 - protein, 1088 aa, Carica papaya
  papaya CpTZP
MNVDKDNLEPHTDLGLTVNYSNHCIQRRLNNHLGAGANAGSSRGMTFVTTDPLSELVWSPQKGPSLKCADCSFSDKKSSLLWDAGPSNVVFSSAPPMISAVRCSNDKLMNENNNMIKSHMGFYVKSDVGDRHTSARCPTNNAAIKPVSGPSPEQKTGTGGHMEETSTILQLSVQHANKNDDLSRPKGEVTCDLNTIQADEAAEAIEDNLKNFLDVLRPNVAQTEPLFENPAGGYCDANSENQTSKIEINLMSDIHAKNKTEACDDALKSQTVPPKRREEPASFTEGENDRKIKSTDSSNIRCMEKLESTAENDLQIHIGENGCDAASKSVATEFAHEGKRCSQQEKARFREKLASGKYSAKNSGIQKYERKGKEKALSDGNLSMMSKEADDSYESVESCNGTGLFSTGKRRWNFEQQMIAGSKRAKMQIRETLESTSFVKQDSSFVNWIGNMMKGFLKLKEDEAPSVSLTLAYPTNGGPDHDTIATTRNKNPGCRTVGFQSIFQSIYCPQRRIQDVTTSNDKCHKEVELPNDIFNPNPVNCHGNFTKQFTISDERNTESTFGNGVLLETQPKISSVNFAASQRTRIPTLQRQMVVQFAQYKGKEGTSSSSSLGKYKLNIAENIDSEPLSEGESGQNFGNGSDHLGSLWIARLAPKTSGFSLNLNQQNRSTDVDLEYSADCAKLPHSPQYHVSSPSEAKIVEARQHSLDDQKIAIGNDLPKCAGETECKITCNSAKDHNDHKSTYNMNPILSSPIENSEAMASLFARRLDTLRHIMPSDLVENTASATVSCFFCGRKGHHLRDCPEMTDEDLEDLLRTVNKYNGTEEFSSFCVRCFRLNHWAVTCPNASSKGNRQRECGASLAGEFSPSSGKHNSRIEENPKFLNGNDSQFQVAEVHSLLDRNDSGIRASLKLNSTEKLVASSSGKNKLRGNEIVPLSKYITRESSDVPKGIFDAVRMLRLSRTDILKWMNSQMAGSHLDGCFLRLRVGKWEKGLGGTGYYVACITEAQRQSSPQSSKNSIFVVVRGIRCLVESQYISNHDFLEDELNAWWFTIEKNGGTIPMEEVLRLKDRHKYNLYIGVVCYVDKKF

SEQ ID NO: 9 - protein, 1067 aa, Ricinus communis
  castor bean RcTZP
MNVDDKNIEPSTNLSLALGYSNQCIQRNLSNDPGAGANAASTADITFVATDPLSELVWSPHKGLSLRCADGSFIDKKPSLLPGVGPTYMASGSSSDKPISNTGKLFDNEICIASLPACKLASEISGDNSTTFLTSNVGIMPLSGTGLDKTATGDQVVEMKNAVNYFLQKEDLRNDKAEDETKLDVAQNYRTFEEPIVRATDVNDDHELGMEIVLVSDFHTVKGREDYGIKIQNAACSGKENEEPPSVREKERKNKMVIGRPGIFSLDKLESTAENDLETPFGENSCSMRNKNLASESADRVENNTQHELIPIEYALGYNQSPTSSRLQNIQRQGQSKALSDGDAKERMLNEEDGSHESVESCNSTELFSTGKQRWNFDQQLIVGSKRVKRQIQDSPGSSSLGKQDSSFVNWISNMMKGFLKSSEGEAPFLSSALSNPNYGHENPSQDVFTCNRKEDPACDTRGFQSVFQSLYCRKTKGQETVTLNVNHQTEGSKECDQDNKICDLNAAPIACRMVTGNVYKRFLPSNEKHNEPTSGYHAGMTVHSRDISMSFPVIPESNGSVSTENKNSCNLAIGKEKDGTDSNFSHGKHKTSSAGKIDPELPSEDKTAHGFGYKGDPLGSLWIARFSPKTSGAPFNHYPSNKSTGEAFNCSADSMGLIPQVQNPLGSSSEHEIVEVRNKNFQEPLPIQNYSTANRAPFDFYNVKGNIDNDSGNKLNPILSSARVKTSEAMASVSPRRLDAPKYITPSDDADNSDRASMTCFFCGIKGHDLRECSEVTDTELEDLLRNINIYGGIKELPCVCIRCFQLNHWAVACPSTCPRVRSKAECHASSVSHAGPSKSQLHVINEDDTKAKNVTGSGHAICYGNDYGMDKDMNSWKSNEAATSGKMKLNIRLFEKNISSTSREKELKENQIIPLYGFVNGLISDVPNGIFDAVRSLRLTRTNILKWMNSSASLSIDGYFVRLRLGKWEEGLGGTGYYVARITGMKSKKSIAVNVGGIQCVIESQFVSNHDFLEDELKAWWSATSKVGGKLPSEKELRLKVEEKNXXGGGGGNKIVIENHIVQYQ

SEQ ID NO: 10 - protein, 959 aa, Brachypodium distachyon
  brachypodium BdTZP
MCPLSELVWSPDDGLSIKIAASSLSTRKASLRWNADTLNIVISSPQQSSGRGKSGDNIDATIADAGEMPSQPRTRCDSSVRLSMASPNRIRNLDAQQSTSVRSQEQDSKCCGGISVMNKGKEVSQSCFVYNADKGEVGSCPTRCCKDVSDGSASRKGVIPSISEKQVYCATTVHNERPWADNAWRARLVKAISQKDYVLPNNAMNAQSASSFEKFGNAEKVAGKLAGFLGKENDNHQDQVMQENHHGNHQDRIVQENHNDSHQYHVMQENHMDEPVLGRCGSASGGNPVSRCESASGVNSAARYESSSGVNPTKLEKGKEKVMHDQSNCVSNIKEGDDSNESMESCQSMKAQKREHAQCSIAEMSSRTKRCRREFNESSCSGFLQRNGSSFFNWVSSLTNGLTVFDKSTTDVSLDQKFSVSTVHEFAEQSGPLQNNSSVPVQSVGFNSLFQSLYRHNVMITSTDTCHQSEKKCTEHEADRVALALNDSNSMLGKQIGTSRKTLDVPTETLAADSLLMDFGGGRGNFQNQIGVFPLRAGRNLMMPNSSKSCSRSLEEKQNEAHAGSLKASVGNKGGFRESLWVSRLLPKTSMKLTDATPCNIKSDFCAVNPKGAADKLYCSSQQNFSVEKEFNNSQYYTSTGSDNGTTSSKCPAIPPEENKQSETMASILAKRLDALRHAKTSAVRLGNSCDQTISKECNHGKSPFVVSYSGHDVQEARHETQKSSSGDGKLVLWLGDKGKGQLCTGSDEEVRVKFLSGGDRQHCGGSMAGKAAAPHDNLEANTSAEYVQRRGVKIKEVLSNSMESLPDNKQIVPYGIMSSDEYDRSSAVFGALERLRLSRSDIIRWLTSPVRHTTLDGFFLRLRFGKWEEALGGTGYHVARINGALDRNRLSVTIRNSTCQVDSRFVSNHEFHEDELKAWWSAAMKGGWKLPSNEELSKKLRERELLHPQNRTGQHNDT

SEQ ID NO: 11 - protein, 827 aa, Sorghum bicolor
  sorghum SbTZP
MSPLSELVWSPDEGLSIKIAASSLSTRKASLRWNADTLSILISSPQHSGSGPGVKSGDTIYDNLEVSEKMPSQLRIRSDSSVRVTMDSPNRVTNVDALQSTSIRSQEQDSSSASRKEVMPSISENQVCCATTVRNERSWATNAWRARLVKAVCQKEPMLSMNTENPMLPSSLGISCDAVEVSGKLVGSQGNRNVQSLGSDNNVISNAPEIPSHNNCQDPVLQESHKDEPVVARGESASGVNAVARCESVPGVNSRKLVKDKEKVLYNDSNYGNNTMEGDDSNESIESCTSTKAPKRKHAQFCAATMPSSGNKRFRREDNESSCSGLLQKCGTSFFNWMSSLTNGLPMLDGATAAIPLDQKFSASTGEGSAAPSQPLQNNSSVPMQSVGFNSLFQSLYTHNVMITSRDNCHQPESNYAGHVFNRLTLELNDSNSMLDKQIGMGRETLDSGCKKNQCTAACSNDATQNKGGLRESLWVTRLLPKASVELMEATPCNVENAVNPQAVGDKLCCPPLQNFNLERGSSDGVTSSKCPATPPEEPKQSETMASVFAKRLDALRHANTSAVRLAIACDRGSPKLRNHKTNSFVVSYSSHDKVEAGHENHKSSSRNGRIVLWLGDKGKEQLCLSNKESRGNFISEHEHQHHGGNTAGKSATRPNLELNTLAEDTDRSQVKLKGGVSDFMAGPSDNKQIVPYGTMPNDVCDESSVVFGALHRLRLSRSDIIRWLRSPIMHTTLDGFFVRLRFGKWEEALGGTGYHVARLNGALDRSRLSVTIRNSTCQVDSRFVSNHDFHEDELKAWWSAAMKSEWKLPSKEELSVKLRERELLRS

SEQ ID NO: 12 - protein, 597 aa, Vitis vinifera
  grape VvTZP
MYRHRTKGKGKALSDGDRSGRKSNKEDDSDESVESCNSAALFSTGKKRWGYEQQLITGSKRIRKQINGSPGSTSFVRQDSSFMSWISNMMKGLSKSNQDETPSLALTLARPNHDNYDQKLVTCNKNQDPGCRNIGFQSIFQSLYCPTTKVQESRTLNADNQTGEGSKEFCLANKLCDFNQSTFGNRAGPSTQPKVLSAKFAVSQENYKTSSVENRSASNPVSSSSSLGKRKANSAENNDSDPPSEGKTIHNFGYKSDLLGSLWVTRFSPKTSSPTCKVDHCNQNTGGATELSTDCMGLIPYSQNRTEVSFGFKKNNAHNNQNSIYKLNPISPSQRFKSSEAMASLFARRLDALKNIITLNQTDTEARATPTCFFCGIRGHSIHDCSEIKETELEDLLRNNNLYPGAEEPPCFCIRCFQLNHWAVACPSVLKRQNQSECGASLVNRCSSESQIIPLCNFVNPQISDVPKGIFDAIKRLRLSRGDILKWMNSVFPFSHLNGFFLRLRLGKWEEGLGGTGYYVACISGAQKERPSQSSKNPIAVNIGGVKCLVQSQYISNHDFLEDELMAWWGATTRAGGKIPSEEDLKVKLEERKKFGF

SEQ ID NO: 13 - protein, 14 aa, Artificial Sequence
  synthetic zinc knuckle (znkn) domain
CXXCXXXXHXXXXC

SEQ ID NO: 14 - protein, 17 aa, Artificial Sequence
  synthetic LIGHT5 Shahdara (Sha) allele amino acids 162-179
RMESAGSQEGPSNRGDK

SEQ ID NO: 15 - protein, 13 aa, Artificial Sequence
  synthetic LIGHT5 Bay-0 (Bay) allele amino acids 162-179
VNEQLSMESAGSQ

SEQ ID NO: 16 - protein, 779 aa, Populus trichocarpa
  poplar Populus2TZP
MEKERDNYMETGPYICPLEKLESTAENDFKTPHSENVCDVATEIVGSQNAKEVRSSSQQDDEILPKDNDCAIKQSPTYSRTRRYQMKGKVKALSDGNLNERMLDMDDDSHEKNDFKTPHSENVCAVATEIVGSQNAKEVRSSSQQDDEILPKDNDCAIKQSPTYSRTRRYQMKGKAKALSDGNLNERMLDMDDDSHESVESCNSVGLFSTGKRQRNFDPHSYVGSKSIKTKIQESPGSSSFVKHDGSFMNWISNMMKGFLKSNEDEAPSLALTLANHKHGHEDRDKNLISCNRNQDQGCKTMGFHSLFQSLYCPKTKAQETVALNANTQTEGSKELGLDNKICDSNATPITCPMVTDNVYKRFLQPNEKLNESTSGNGAASPALTKLLSTNIASSQEISGSNSAEKKNSCNMATDKEKNGTSSNSSPGKRKMNDAEQPSEGKATNTSGYRSDPLTSLWITRLSPKTSGPLSNRDLCHRRTGEALDGFTDFIRLKAQWQNHPSSYQDKNIVGAREEEHFTEDPVCMHNCANSTEVSFSINKVNGHHDEKSMCKMNSTLPFSRFRNSEAMASVFARRLDALMHIMPSYGTDDSSHGNLTCFFCGIKCHHVRDCPEIIDSELADILRNANSFNGANEFPCVCIRCFQSNHWAVACPSASSRTRHQAEYGASLVHESSPCKILLNPRNEDDAKQSDGKDSQLQAADAPTVRNGKLHEASASGKINMNMKPFERDTASSSGEKKLKENQVMPLSNFINSQIADVPKGIFDAVKRLRLSRTIILK

SEQ ID NO: 17 - protein, 941 aa, Glycine max
  soybean Gm3TZP
MSAENEKIKPKTDIELFLNNANQCIWKKLNNDSGAGANAASRADMTLAATDPLSEIVWSPDKGLSLKCADSSFAHKNSSLLRDVGTSCMVFAPPQNFTGGSSTTDKPLDDDFLKPIAVVCAKSDIAEADAPTMPPTGDSGVKAKCKAYEEDDIGSVGNKEKVNTAATAPNLPNEQNGNLTNNWEKITGDQANSGTDKVSGIEGNRISAISEFFFCCSGPTCDVSFSVHRHSFVNSNGEGQADQGPFDHLLLQSDENKPSMDQNPSPGRHSDGSVNIGLEKKAVVTDDDLHTAVEPIIEYRGSGAHETNLASSSKNPLEKLEYSAENDLQTFNCEAACAGTSRVNVSETENKFQDTEMMLPCDKILPVLHSPCHSRIHMAINKGKEKSLSDGDANVMLSREENDSHSSVESCNSTGFFSTGKKRRNFQQQLIIGSKRVKKQIEESSGPKSYVKQDNSFMNWISNMVKGLSPSIQNDSNTLALTLANPDHHNLQPDEKLIACNMNQDPEPKNTGFKSIFQSICCPSLKNVGTRMSHQEGKSSQDLVPGNMEHGIDATPITCWAENNSLSKLCLQSNKFEVSTGGNDAGLSSQPKIKPLNFFNCHESSKNNPVETKNYSILGHSKDKEEVASHSSSTKQNTDNNDNIDSNVLCDRKEEENICHRRDNLGSLWITRFSPKFTAPLREQPANDTEVSTDLKEDKGNNDHKSMYLFKPLSSSPGFRNLEPTASMFGRRFGAIKQIIPTNATDTTTQVNMLCFFCGTRGHQLSDCLAIAENKLEDLQKNIDSYGGLEEHHCLCIKCFQPNHWAISCPTSISTRKHELKANALVNDCGKHLISSNEGSARLLTDEDDRVLSGGSINDETDQRAGQNINLKWKSNEIITHKVGCNASFKKYRGLSSEENKFRENPTLSPSKLAERQISQVPKEIFEAVKKLQLSRTDILK

SEQ ID NO: 18 - protein, 1071 aa, Carica papaya
  papaya CpTZP2
MNVDKDNLEPHTDLGLTVNYSNHCIQRRLNNHLGAGANAGSSRGMTFVTTDPLSELVWSPQKGPSLKCADCSFSDKKSSLLWDAGPSNVVFSSAPPMISAVRCSNDKLMNENNNMIKSHMGFYVKSDVGDRHTSARCPTNNAAIKPVSGPSPEQKTGTGGHMEETSTILQLSVQHANKNDDLSRPKGEVTCDLNTIQADEAAEAIEDNLKNFLDVLRPNVAQTEPLFENPAGGYCDANSENQTSKIEINLMSDIHAKNKTEACDDALKSQTVPPKRREEPASFTEGENDRKIKSTDSSNIRCMEKLESTAENDLQIHIGENGCDAASKSVATEFAHEGKRCSQQEKARFREKLASGKYSAKNSGIQKYERKGKEKALSDGNLSMMSKEADDSYESVESCNGTGLFSTGKRRWNFEQQMIAGSKRAKMQIRETLESTSFVKQDSSFVNWIGNMMKGFLKLKEDEAPSVSLTLAYPTNGGPDHDTIATTRNKNPGCRTVGFQSIFQSIYCPQRRIQDVTTSNDKCHKEVELPNDIFNPNPVNCHGNFTKQFTISDERNTESTFGNVREQEYQLFRDKWLCNLHNSKGKEGTSSSSSLGKYKLNIAENIDSEPLSEGESGQNFGNGSDHLGSLWIARLAPKTSGFSLNLNQQNRSTDVDLEYSADCAKLPHSPQYHVSSPSEAKIVEARQHSLDDQKIAIGNDLPKCAGETECKITCNSAKDHNDHKSTYNMNPILSSPIENSEAMASLFARRLDTLRHIMPSDLVENTASATVSCFFCGRKGHHLRDCPEMTDEDLEDLLRTVNKYNGTEEFSSFCVRCFRLNHWAVTCPNASSKGNRQRECGASLAGEFSPSSGKHNSRIEENPKFLNGNDSQFQVAEVHSLLDRNDSGIRASLKLNSTEKLVASSSGKNKLRGNEIVPLSKYITRESSDVPKGIFDAVRMLRLSRTDILKWMNSQMAGSHLDGCFLRLRVGKWEKGLGGTGYYVACITEAQRQSSPQSSKNSIFVVVRGIRCLVESQYISNHDFLEDELNAWWFTIEKNGGTIPMEEVLRLKDRHKYNLYIGVVCYVDKKF

SEQ ID NO: 19 - protein, 100 aa, Artificial Sequence
  synthetic Brachypodium distachyon TZP (BdTZP) PLUS3 domain
SAVFGALERLRLSRSDIIRWLTSPVRHTTLDGFFLRLRFGKWEEALGGTGYHVARINGALDRNRLSVTIRNSTCQVDSRFVSNHEFHEDELKAWWSAAMK

SEQ ID NO: 20 - protein, 100 aa, Artificial Sequence
  synthetic Sorghum bicolor TZP (SbTZP) PLUS3 domain
SVVFGALHRLRLSRSDIIRWLRSPIMHTTLDGFFVRLRFGKWEEALGGTGYHVARLNGALDRSRLSVTIRNSTCQVDSRFVSNHDFHEDELKAWWSAAMK

SEQ ID NO: 21 - protein, 100 aa, Artificial Sequence
  synthetic Oryza sativa TZP (OsTZP) PLUS3 domain
SVVFGALQRLRLSRSDIIRWMRSPVMHTTLDGFFLRLRFGKWEEALGGTGYHVARINGVLDKNRLSVTIRNSTCQVDSRFVSNHDFHQDELKAWWSAAMK

SEQ ID NO: 22 - protein, 109 aa, Artificial Sequence
  synthetic Arabidopsis thaliana TZP (AtTZP) PLUS3 domain
RAVFDAVQVLRLSRTDVLKWINTKKSVSGLEGFFLRLRLGKWEEGLGGTGYYVARIDGDTEGQSSRRHSEKSLISVKVKGVTCLVESQFISNQDFLEEELKAWWQSAGK

SEQ ID NO: 23 - protein, 109 aa, Artificial Sequence
  synthetic consensus of 96 Arabidopsis thaliana TZP accessions (AtTZPaccessions) PLUS3 domain
KAVFDAVQGLRLSRTDVLKWINTKKSVSGLKGFFLRLRLGKWEEWLGGTGYYVARIDGDTEGQSSRRHSERSLISVKVKGVTCLVESQFISNQHFLEEELKAWWQSAGK

SEQ ID NO: 24 - protein, 109 aa, Artificial Sequence
  synthetic Arabidopsis lyrata TZP (AlTZP) PLUS3 domain
KAVFEAVQVLRLSRTDVLKWINTKKSVSGLEGFFLRLRLGKWEEGLGGTGYYVARIDGDTEGQSSRRPSEKSSISVKVKGVTCLVESQFISNHDFLEEELKAWWRSAGK

SEQ ID NO: 25 - protein, 104 aa, Artificial Sequence
  synthetic Populus trichocarpa TZP (PtTZP) PLUS3 domain
FDAVKMLRLSRAVILKLMDSHAAPSRLDGFFLRLRLGKWEQGLGGTGYYVACITGVESESSTQKSKNSIAVNVGGIKYLVESQCISNHDFTEDELMAWWRATLK

SEQ ID NO: 26 - protein, 104 aa, Artificial Sequence
  synthetic Vitis vinifera TZP (VvTZP) PLUS3 domain
FDAIKRLRLSRGDILKWMNSVFPFSHLNGFFLRLRLGKWEEGLGGTGYYVACISGAQKERPSQSSKNPIAVNIGGVKCLVQSQYISNHDFLEDELMAWWGATTR

SEQ ID NO: 27 - protein, 104 aa, Artificial Sequence
  synthetic Carica papaya TZP (CpTZP) PLUS3 domain
FDAVRMLRLSRTDILKWMNSQMAGSHLDGCFLRLRVGKWEKGLGGTGYYVACITEAQRQSSPQSSKNSIFVVVRGIRCLVESQYISNHDFLEDELNAWWFTIEK

SEQ ID NO: 28 - protein, 101 aa, Artificial Sequence
  synthetic Ricinus communis TZP (RcTZP) PLUS3 domain
VPNGIFDAVRSLRLTRTNILKWMNSSASLSIDGYFVRLRLGKWEEGLGGTGYYVARITGMKSKKSIAVNVGGIQCVIESQFVSNHDFLEDELKAWWSATSK

SEQ ID NO: 29 - protein, 109 aa, Artificial Sequence
  synthetic Glycine max TZP (GmTZPa) PLUS3 domain
KKIFDAVKKLQLSRTDILKCINTHGSISQLDGFFLRLRLGKWEEGLGGTGYHVAYINETQSQRQCPEQNTRKCLSVKVGSIKCMVESQYISNHDFLEEEITEWWSNTSE

SEQ ID NO: 30 - protein, 109 aa, Artificial Sequence
  synthetic Glycine max TZP (GmTZPb) PLUS3 domain
KEIFEAVKKLQLSRTDILKWINTRGSISQLDGFFLRLRLGKWEEGPGGTGYHVAYINETQSQRQCSEQNTRKSLSVKVGSIKCMVESQYISNHDFLEEEIMEWWSNTSQ

SEQ ID NO: 31 - protein, 109 aa, Artificial Sequence
  synthetic Selaginella moellendorffii TZP (SmTZP) PLUS3 domain
KDMLAAIESVRLSRSELWRWMDSPDFNTNVRGFFLRLRFGRWEKDLGGTGYRMARIRGAIRKLGGDDKNMSVSVDLGDMECTVDSQYVSNHSFTEGSKDLSSLWIVLCS

SEQ ID NO: 32 - protein, 111 aa, Artificial Sequence
  synthetic Arabidopsis thaliana TZP (At3g51120) PLUS3 domain
AIDVHNINLIYLRRKFLESLLDDINKVDEKVVGTILRIKVSGSDQKLDIHRLVQVVGTSKAIASYQLGAKTTDVMLEILNLDKREVISIDQLSDQNITEDECKRLRQSIKC

SEQ ID NO: 33 - protein, 143 aa, Artificial Sequence
  synthetic Arabidopsis thaliana TZP (AT2G16480) PLUS3 domain
PNHVDVDENLDHPVKSGKDKKRKTRKKNVRKGRQSNLDDFAAVDMHNINLIYLRRSLVEDLLEDSTAFEEKVASAFVRLRISGNQKQDLYRLVQVVGTSKAPEPYKVGKKTTDYVLEILNLDKTEVISIDIISNQDFTEVTYC

SEQ ID NO: 34 - protein, 103 aa, Artificial Sequence
  synthetic Arabidopsis thaliana TZP (At5g08430) PLUS3 domain
AIVSDNIKLLYLRKSLVQELLKSPDTFEGKMLGSFVRIKSDPNDYLQKYPYQLVQVTGVKKEHGTDDFLLQVTNYVKDVSISVLSDDNFSQEECEDLHQRIKN

SEQ ID NO: 35 - protein, 101 aa, Artificial Sequence
  synthetic Arabidopsis thaliana TZP (At5g63700) PLUS3 domain
TINADNLKLVYLRKSLVLELLKQNDSFVDKVVGSFVKVKNGPRDFMAYQILQVTGIKNADDQSEGVLLHVSGMASGVSISKLDDSDIREEEIKDLKQKVMN

SEQ ID NO: 36 - protein, 113 aa, Artificial Sequence
  synthetic Arabidopsis thaliana TZP (AtVIP5) PLUS3 domain
VPTFEDVKEVTIRRSKLAKWLMEPFFEELIVGCFVRVGIGRSKSGPIYRLCWVKNVDATDPDKTYKLENKTTHKYLNVVWGNETSAARWQMAMISDGHPLEEEYRQWIREVER

SEQ ID NO: 37 - protein, 759 aa, Arabidopsis lyrata
  Al TZP
MGDGDEQSKELEGLSSSSRRSSGPSSANAEARMKFASVDAITELVWSPGNGLSLRCADISFTGKAKLVSPNFFDIGLTNMAIHSNSTSIEHQEDVELRSRDQVNQERIGGSVEDMKPEMVEDKVETDDDIKNEVAGSSKRSSDSPKVMEGETRDLLVNEQLRMESAGSQEGPTNRGDKDEGDKANNRIDRLESMDENNLATLAVVACEGQGEYSLENEAGPSGSYRRPKQDSSFMNWISNMTKGIWKGNEEDDSPFAALTTTSDANGHGQVNAIVDQQQLSPCCVKENSGCRNTGFQSLFQSIYCPKKRSQDAVEMDFPNDANATSLQELPWIPEQCGIAKGDDLSSSDNDIGPVAEPNISSGKVGFNQRSETLSSENKREDKEPNISLMSLSKSKPNEEPKICGEAGGKVSPCLNNRNSGLQSLWISRFSSKSPFPQKKTSETAKEVNASASDTAKTHDSQKMLVNNNVVIPSISSVDGLDKLNTVLPIVSSMRIESSEAMASLFARRLEAMKHIIPAGSLAENAEEEQPNLICFYCGKKGHCLQDCLEVTDTELRDLVQNISSRNGREEASSLCIRCFQLSHWAATCPNGPLYSSGAEDRAMKHTLASTSGMKLPVSGFTDVPKAVFEAVQVLRLSRTDVLKWINTKKSVSGLEGFFLRLRLGKWEEGLGGTGYYVARIDEGQSSRRPSEKSSISVKVKGVTCLVESQFISNHDFLEEELKAWWRSAGKSAERSGCEGIPSAEELSRKIQQRKMLNR

(19) United States
(12) Patent Application Publication
(10) Pub. No.: US 2012/0017335 A1
(43) Pub. Date: Jan. 19, 2012

(54) ZINC KNUCKLE PROTEINS

(75) Inventors: Olivier Loudet, Bazainville (FR); Todd P. Michael, Piscataway, NJ (US); Detlef Weigel, Tuebingen (DE); Joanne Chory, Del Mar, CA (US)

(73) Assignee: The Salk Institute for Biological Studies, La Jolla, CA (US)

(21) Appl. No.: 13/123,234

(22) PCT Filed: Oct. 9, 2009
(86) PCT No.: PCT/US09/60232

§ 371 Date: Oct. 4, 2011

Related U.S. Application Data
(60) Provisional application No. 61/195,819, filed on Oct. 10, 2008.

(51) Int. Cl. C12N 15/82; C12N 15/63; A01H 5/00

(52) U.S. Cl. 800/287; 800/298; 435/320.1; 800/288

(57) ABSTRACT

TZP proteins and method of their use for improving plant characteristics are disclosed.

### CROSS-REFERENCE TO RELATED PATENT APPLICATIONS

The present application claims benefit of priority to U.S. Provisional Patent Application No. 61/195,819, filed Oct. 10, 2008, which is incorporated by reference for all purposes.

### STATEMENT AS TO RIGHTS TO INVENTIONS MADE UNDER FEDERALLY SPONSORED RESEARCH AND DEVELOPMENT

This invention was made with government support under (identify the contract) awarded by the National Institutes of Health (NIH Grant No. GM62932). The government has certain rights in the invention.

### BACKGROUND OF THE INVENTION

The embryonic stem or hypocotyl is an excellent model for studying both internal and external factors controlling growth in plants (Nozue, K. et al., Nature, 448:358-361 (2007)). Genetic screens in common laboratory accessions have yielded direct molecular insight into how light and hormone dependent signaling pathways interact with the circadian clock to regulate the final length of the hypocotyl (Nozue, K. et al., Nature, 448:358-361 (2007)). The power of the hypocotyl assay is its simplicity, as well as its obvious meaningfulness. When germinating seeds are exposed to low levels of light, such as those caused by a covering layer of debris, the hypocotyl has to grow for a while. Only after the surface has been broken by the tip of the hypocotyls can the embryonic leaves, the cotyledons, unfold. Conversely, if a seed has fallen on open ground, there is no need for the hypocotyls to be particularly long. Because of the ease and reproducibility with which hypocotyl length can be measured in thousands of individuals, it has also been a powerful model in mapping genes with more subtle effects on light and hormone regulated growth, using methods of quantitative genetics (Borevitz, J. O. et al., Genetics, 160:683-696 (2002)). Multiple light signaling genes controlling hypocotyl length have been characterized in quantitative trait locus (QTL) studies (Aukerman, M. J. et al., Plant Cell, 9:1317-1326 (1997); Balasubramanian, S. et al., Nat Genet, 38:711-715 (2006); Filiault, D. L. et al., Proc Natl Acad Sci USA, 105:3157-3162 (2008); Maloof, J. N. et al., Nat Genet, 29:441-446 (2001)).

### BRIEF SUMMARY OF THE INVENTION

The present invention provides for plants comprising a heterologous expression cassette comprising a promoter operably linked to a polynucleotide encoding a TZP polypeptide. In some embodiments, the promoter is heterologous to the polynucleotide. In some embodiments, the plant is characterized by increased size, speed of growth, elongation of organs or delay or extension of time of seed and/or fruit production compared to a plant lacking the heterologous expression cassette. In some embodiments, the promoter is a tissue-specific promoter. In some embodiments, the TZP polypeptide is at least 50% (e.g., 60%, 70%, 80%, 90%, 95%, 98%, 99%) identical to SEQ ID NO:2, SEQ ID NO:4, SEQ ID NO:5, SEQ ID NO:6, SEQ ID NO:7, SEQ ID NO:8, SEQ ID NO:10, SEQ ID NO:11, SEQ ID NO:12, SEQ ID NO:16, SEQ ID NO:17, or SEQ ID NO:18. In addition to the above described plants, the present invention also provides for processed plant material, including but not limited to processed plant parts (e.g., seeds, fruit, nuts, tubers, leaves, etc.). The present invention further provides a method of making processed plant material, the method comprising processing a plant or plant part of the present invention, thereby generating processed plant material.

The present invention also provides for isolated nucleic acids comprising a promoter operably linked to a polynucleotide encoding a TZP polypeptide wherein the promoter is heterologous to the polynucleotide. In some embodiments, the TZP polypeptide is at least 50% (e.g., 60%, 70%, 80%, 90%, 95%, 98%, 99%) identical to SEQ ID NO:2, SEQ ID NO:4, SEQ ID NO:5, SEQ ID NO:6, SEQ ID NO:7, SEQ ID NO:8, SEQ ID NO:10, SEQ ID NO:11, SEQ ID NO:12, SEQ ID NO:16, SEQ ID NO:17, or SEQ ID NO:18.

The present invention also provides for methods for increasing size, speed of growth or biomass in a plant. In some embodiments, the method comprises, introducing an expression cassette into one or more plants, the expression cassette comprising a promoter operably linked to a polynucleotide encoding a TZP polypeptide, and selecting a plant that has increased size, speed of growth, elongation of organs or delay or extension of time of seed and/or fruit production compared to a plant lacking the heterologous expression cassette. In some embodiments, the TZP polypeptide is at least 50% (e.g., 60%, 70%, 80%, 90%, 95%, 98%, 99%) identical to SEQ ID NO:2, SEQ ID NO:4, SEQ ID NO:5, SEQ ID NO:6, SEQ ID NO:7, SEQ ID NO:8, SEQ ID NO:10, SEQ ID NO:11, SEQ ID NO:12, SEQ ID NO:16, SEQ ID NO:17, or SEQ ID NO:18.

### DEFINITIONS

The term “promoter” or “regulatory element” refers to a region or sequence determinants located upstream or downstream from the start of transcription and which are involved in recognition and binding of RNA polymerase and other proteins to initiate transcription. Promoters need not be of plant origin, for example, promoters derived from plant viruses, such as the CaMV35S promoter, can be used in the present invention.

The term “plant” includes whole plants, shoot vegetative organs/structures (e.g. leaves, stems and tubers), roots, flowers and floral organs/structures (e.g. bracts, sepals, petals, stamens, carpels, anthers and ovules), seed (including embryo, endosperm, and seed coat) and fruit (the mature ovary), plant tissue (e.g. vascular tissue, ground tissue, and the like) and cells (e.g. guard cells, egg cells, trichomes and the like), and progeny of same. The class of plants that can be used in the method of the invention is generally as broad as the class of higher and lower plants amenable to transformation techniques, including angiosperms (monocotyledonous and dicotyledonous plants), gymnosperms, ferns, and multicellular algae. It includes plants of a variety of ploidy levels, including aneuploid, polyploid, diploid, haploid and hemizygous.

A polynucleotide sequence is “heterologous to” a second polynucleotide sequence if it originates from a foreign species, or, if from the same species, is modified by human action from its original form. For example, a promoter operably linked to a heterologous coding sequence refers to a coding sequence from a species different from that from which the promoter was derived, or, if from the same species, a coding sequence which is different from naturally occurring allelic variants.

An “expression cassette” refers to a nucleic acid construct, which when introduced into a host cell, results in transcription and/or translation of a RNA or polypeptide, respectively. Antisense constructs or sense constructs that are not or cannot be translated are expressly included by this definition.

An “TZP polypeptide” is a polypeptide substantially identical to SEQ ID NO:2, SEQ ID NO:4, SEQ ID NO:5, SEQ ID NO:6, SEQ ID NO:7, SEQ ID NO:8, SEQ ID NO:10, SEQ ID NO:11, SEQ ID NO:12, SEQ ID NO:16, SEQ ID NO:17, or SEQ ID NO:18 or as otherwise described herein. TZP polypeptides comprise a tandem (e.g., two) zinc knuckle domains (CX₂CX₄HX₄C; SEQ ID NO:13) as well as a PLUS3 domain. Exemplary TZP genomic and coding sequences are shown in SEQ ID NO:1 and SEQ ID NO:3, respectively.

Optimal alignment of sequences for comparison may be conducted by the local homology algorithm of Smith and Waterman Add. APL. Math. 2:482 (1981), by the homology alignment algorithm of Needle man and Wunsch J. Mol. Biol. 48:443 (1970), by the search for similarity method of Pearson and Lipman Proc. Natl. Acad. Sci. (U.S.A.) 85: 2444 (1988), by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” of polypeptide sequences means that a polypeptide comprises a sequence that has at least 25% sequence identity. Alternatively, percent identity can be any integer from 25% to 100%. Exemplary embodiments include at least: 25%, 30%, 35%, 40%, 45%, 50%, 55%, 60%, 65%, 70%, 75%, 80%, 85%, 90%, 95%, or 99%. compared to a reference sequence using the programs described herein; preferably BLAST using standard parameters, as described below. Accordingly, TZP sequences of the invention include nucleic acid sequences encoding a polypeptide that has substantial identity to SEQ ID NO:2, SEQ ID NO:4, SEQ ID NO:5, SEQ ID NO:6, SEQ ID NO:7, SEQ ID NO:8, SEQ ID NO:10, SEQ ID NO:11, or SEQ ID NO:12. TZP sequences of the invention also include polypeptide sequences having substantial identify to SEQ ID NO:2, SEQ ID NO:4, SEQ ID NO:5, SEQ ID NO:6, SEQ ID NO:7, SEQ ID NO:8, SEQ ID NO:10, SEQ ID NO:11, or SEQ ID NO:12. One of skill will recognize that these values can be appropriately adjusted to determine corresponding identity of proteins encoded by two nucleotide sequences by taking into account codon degeneracy, amino acid similarity, reading frame positioning and the like. Polypeptides which are “substantially similar” share sequences as noted above except that residue positions which are not identical may differ by conservative amino acid changes. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, aspartic acid-glutamic acid, and asparagine-glutamine.

Another indication that nucleotide sequences are substantially identical is if two molecules hybridize to each other, or a third nucleic acid, under stringent conditions. Stringent conditions are sequence dependent and will be different in different circumstances. Generally, stringent conditions are selected to be about 5° C. lower than the thermal melting point (Tm) for the specific sequence at a defined ionic strength and pH. The Tm is the temperature (under defined ionic strength and pH) at which 50% of the target sequence hybridizes to a perfectly matched probe. Typically, stringent conditions will be those in which the salt concentration is about 0.02 molar at pH 7 and the temperature is at least about 60° C.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

### BRIEF DESCRIPTION OF THE DRAWINGS

FIG. 1. Confirmation and fine-mapping of the LIGHT5 QTL to three genes, among which At5g43630 is highly polymorphic between Bay-0 and Shandara. (A) Confirmation of LIGHT5 using heterogeneous inbred families (HIFs). The Sha allele is fully dominant over the Bay allele. Two rounds of recombinant screening from HIF350 followed. (B) Horizontal marks on the chromosomes are markers with physical position in Mb indicated to the right. Red arrows indicate the approximate position of different recombination events that were individually tested in rHIF to establish the QTL position. Fine-mapping identified a 7 kb-region containing three genes. (C) Grey boxes along the vertical axes represent exons from three genes highlighted by vertical arrows. Horizontal red arrows indicate the exact physical position of the last 5 recombinants defining the QTL candidate region. Amino-acid changes between Bay-0 and Shandara within the interval are presented in the table (RMESAGSQEGPSNRGDK=SEQ ID NO:14; VNEQLSMESAGSQ=SEQ ID NO:15) and linked to their respective physical position along the gene model. (D) Model of the protein structure of TZP (At5g43630).

FIG. 2. LIGHT5/TZP controls growth throughout development. (A) Increased TZP activity results in longer hypocotyls under blue light. Seedlings were grown at the fluence indicated and measured on the sixth day. TZP-OX(3) and TZP-OX(5) are two independent transgenic lines overexpressing TZP in rHIF138-8 background. rHIF138-8 contains the Bay allele with the premature STOP in TZP and rHIF138-13 contains the functional Sha allele. (B) Sha allele of TZP or overexpression result in longer hypocotyls. Plants were grown under light/dark cycles (12 hrs/12 hrs) at 22° C. and hypocotyls were measured 7 DAG. Representative seedlings were used to make the images in (C). Measurements represent three independent experiments of twenty seedlings each. (C) TZP-OXplants have long hypocotyls 7 DAG under light/dark cycles (12 hrs/12 hrs) compared to its background (rHIF138-8). (D) TZP-OXpetioles are elongated 24 DAG compared to rHIF138-8. (E) Mature TZP-OXplants are almost twice as tall as background plants 50 DAG. Plants were grown under long days (light/dark: 16 hrs/8 hrs) for 50 days. All plants pictured are representative of at least two independent transgenic lines (lines 3 and 5) and two independent experiments. The vertical bar represents 20 cm. (F) TZP::YFP is localized to speckles in the nucleus. T2 plants carrying the 35S::TZP:YFP fusion were imaged to detect TZP localization. TZP:YFP localizes to the nucleus in guard cells of the stomata, in addition to all other tissues tested. Grey lines highlight the cell walls.

FIG. 3. LIGHT5/TZP controls morning-specific growth pathways. (A) TZP displays dawn-specific transcript abundance under light/dark cycles (12 hrs/12 hrs) and constant 22° C. (six time points). The second day of data is copied from the first (double plotted) for visualization purposes. Expression was determined by qPCR with primers specific to TZP and SyberGreen. (B) TZP transcript abundance is overexpressed in TZP-OX Five independent lines overexpressing TZP were characterized. Two lines are shown here. Data were collected and plotted as in (A). (C) The genes that are misexpressed (P<0.01) in long hypocotyl genotypes (TZP-OX or rHIF138-13 vs. rHIF138-8) under blue light short day photocycles are expressed at dawn as determined with PHASER. (D) Long hypocotyls of the TZP-OX mutantare due in part to overexpression of cell wall genes. As an example of the expression pattern of the cell wall genes that are overexpressed in the TZP-OX mutant, IRX1 continues to cycle with peak expression at dawn, but its peak expression is 3 fold higher in TZP-OX. Other genes that are overexpressed in TZP-OX are listed in table S3 and S10 from our website data at www.inra.fr/vast/Files/Loudet_PNAS_SITables.xls. Results are from array data.

FIG. 4. Phenotypic variation and significant QTL detected in the Bay-0×Shandara RIL set. Distribution of phenotypic values for hypocotyl elongation among 164 Bay-0×Shandara RILs across four different environments: two temperature×two white-light conditions (A). ‘B’ and ‘S’ above bars indicate phenotypic values obtained for the parents, Bay-0 and Shandara respectively. Position and effect of the five significant QTL controlling this variation (B). Each QTL is depicted by a triangle located at the most probable QTL position on one of the 5 chromosomes. Upward- and downward-pointing triangles represent QTLs with a positive or negative allelic effect, respectively (‘2a’ in table S1 from www.inra.fr/vast/Files/Loudet_PNAS_SITables.xls, which represents the mean effect of the replacement of both Shandara alleles by Bay-0 alleles at the QTL). The framework genetic map (horizontal marks indicate marker positions) is from Loudet et al. (2002). QTLs denoted as “LIGHT” were detected in all four environments. Other QTLs are named according to the specific environment in which they had a significant effect.

FIG. 5. LIGHT5 is At5g43630 and the causal polymorphism is the 8 bp-insertion in Bay-0. Two rHIFs segregating for LIGHT5 and defining the limits of the candidate interval were crossed to generate an advanced rHIF (arHIF47) segregating solely for the 7 kb candidate region. Phenotypes are shown for the arHIF47 progeny fixed for either the Bay or Sha 7-kb region. Bay-0[41AV] lacks the 8 by insertion causing the early stop in At5g43630, with the rest of the genome being the same as in Bay-0. Phenotypes are shown from F2 plants between the two isogenic parents. Different letters on bars indicate significantly different means (P<0.01; least significant difference test).

FIG. 6. The PLUS3 domain is conserved across species. (A) Alignment of the PLUS3 domain in multiple species (SEQ ID NOS:19-22 and 24-36) and the consensus of 96 Arabidopsis thaliana accessions (AtTZPaccessions=SEQ ID NO:23).

(B) Phylogenetic tree of the PLUS3 domain across species and with other PLUS3 domains of Arabidopsis thaliana. The TZP PLUS3 domain is more closely related to the PLUS3 domain from TZP orthologs in other species, than other PLUS3 domains in Arabidopsis thaliana. Arabidopsis lyrata (Al), Carica papaya (Cp), Oryza sativa (Os), Ricinus communis (Rc), Populus trichocarpa (Pt), Sorghum bicolor (Sb), Brachypodium distachyon (Bd), Glycine max (Gm), Vitis vinifera (Vv) and Selaginella moellendorffii (Sm).

FIG. 7. TZP expression is correlated with increased hypocotyl growth. Plants were grown under continuous blue light for five days and tissue was collected at dawn. Expression was measured by qPCR. Expression of TZP was higher in lines with longer hypocotyls, including TZP-OX (3), rHIF138-13, arHIF47-5 and Shandara. The bar for TZP-OX (3) was truncated due to the expression being on a different scale than the rest of the lines.

FIG. 8. TZP transcript abundance peaks at dawn in the rHIF and the accession Bay-0 and Shandara. Plants were grown for seven days under light/dark cycles and sampled over one day. Expression was measured using qPCR.

FIG. 9. TZP expression is disrupted in core circadian clock mutants. The expression of TZP was measured in two circadian clock mutants, late elongated hypocotyl (lhy) and early flowering 3 (ems-7) under short day photocycles (8 hrs light/16 hrs dark at 22° C.).

FIG. 10. Core circadian clock gene expression is not disrupted in TZP-OX. Both CIRCADIAN CLOCK ASSOCIATED 1 (CCA1) and GIGANTEA (G1) display completely wild type expression in TZP-OX as compared to HIF138-8.

FIG. 11. Genes upregulated under light/dark cycles in TZP-OX are expressed at dawn. Normally, the 117 genes that are upregulated by TZP-OX underlight/dark cycles are phased to dawn. Overrepresentation plot created using the PHASER tool.

FIG. 12. TZP overexpression specifically disrupts light-specific, time-of-day growth pathways.

### (A) PEROXIDASE30

### (B) DFL1

### (C) HAT4

### (D) CATALASE3 (CAT3)

### (E) PW9 (MATH/TRAF)

(F) SWI/PLUS3 (At2g16480)

### (G) PIF5

### (H) PIF4

### (I) HFR1

### (J) PKS1

### (K) PHYB

### (L) PHYD

FIG. 13. TZP is conserved among species. A. Alignment of full-length TZP sequences from different plant species obtained by BLAST searches using Phytozome (SEQ ID NOS:37, 2, 10, 9, 8, 6, 7, 11, 5 and 12, respectively). The conservation scoring is performed by PRALINE. Increased sequence similarity and identity is observed near the C-terminus of the protein, where the PLUS3 domain resides. B. Phylogenetic tree of TZP protein sequence across species was performed based on Clustal W alignment using MegAlign: Arabidopsis thaliana (At), Arabidopsis lyrata (Al), Oryza sativa (Os), Populus trichocarpa (Pt), Glycine max (Gm), Brachypodium distachium (Bd), Vitis vinifera (Vv), Carica papaya (Cp), Selanginella moellondorfii (Sm), Ricinus communis (Rc).

### DETAILED DESCRIPTION

### I. Introduction

The present invention provides for methods of improving plant traits including but not limited to increasing plant organ elongation, increasing plant height, delaying seed or fruit set, increasing the time of fruit or seed set, and/or increasing plant size. In some embodiments of the invention, a TZP polypeptide is ectopically or otherwise overexpressed. In some embodiments, for example, the invention provides for transgenic plants comprising a heterologous expression cassette for expressing a TZP polypeptide in the plant.

Alternatively, the present invention provides for plants with shortened stature. In these embodiments, endogenous TZP expression is reduced, e.g., by use of siRNA, antisense, or other gene expression reduction technology.

### II. Use of Nucleic Acids of the Invention to Enhance Gene Expression

Isolated nucleic acid sequences encoding all or part of a TZP polypeptide can be used to prepare expression cassettes that enhance, or increase endogenous, TZP gene expression. Where overexpression of a gene is desired, the desired gene from a different species may be used to decrease potential sense suppression effects.

Any of a number of means well known in the art can be used to increase TZP activity in plants. Any organ can be targeted, such as shoot vegetative organs/structures (e.g. leaves, stems and tubers), roots, flowers and floral organs/structures (e.g. bracts, sepals, petals, stamens, carpels, anthers and ovules), seed (including embryo, endosperm, and seed coat) and fruit. Alternatively, one or several TZP genes can be expressed constitutively (e.g., using the CaMV 35S promoter).

One of skill will recognize that the polypeptides encoded by the genes of the invention, like other proteins, have different domains which perform different functions. Thus, the gene sequences need not be full length, so long as the desired functional domain of the protein is expressed.

### III. Preparation of Recombinant Vectors

To use isolated sequences in the above techniques, recombinant DNA vectors suitable for transformation of plant cells are prepared. Techniques for transforming a wide variety of higher plant species are well known and described in the technical and scientific literature. See, for example, Weising et al. Ann. Rev. Genet. 22:421-477 (1988). A DNA sequence coding for the desired polypeptide, for example a cDNA sequence encoding a full length protein, will preferably be combined with transcriptional and translational initiation regulatory sequences which will direct the transcription of the sequence from the gene in the intended tissues of the transformed plant.

For example, for overexpression, a plant promoter fragment may be employed which will direct expression of the gene in all tissues of a regenerated plant. Such promoters are referred to herein as “constitutive” promoters and are active under most environmental conditions and states of development or cell differentiation. Examples of constitutive promoters include the cauliflower mosaic virus (CaMV) 35S transcription initiation region, the 1′- or 2′-promoter derived from T-DNA of Agrobacterium tumafaciens, and other transcription initiation regions from various plant genes known to those of skill.

Alternatively, the plant promoter may direct expression of the polynucleotide of the invention in a specific tissue (tissue-specific promoters) or may be otherwise under more precise environmental control (inducible promoters). Examples of tissue-specific promoters under developmental control include promoters that initiate transcription only in certain tissues, such as fruit, seeds, or flowers. Examples of environmental conditions that may affect transcription by inducible promoters include anaerobic conditions, elevated temperature, or the presence of light.

If proper polypeptide expression is desired, a polyadenylation region at the 3′-end of the coding region should be included. The polyadenylation region can be derived from the natural gene, from a variety of other plant genes, or from T-DNA.

The vector comprising the sequences (e.g., promoters or coding regions) from genes of the invention can optionally comprise a marker gene that confers a selectable phenotype on plant cells. For example, the marker may encode biocide resistance, particularly antibiotic resistance, such as resistance to kanamycin, G418, bleomycin, hygromycin, or herbicide resistance, such as resistance to chlorosluforon or Basta.

In some embodiments, TZP nucleic acid sequences of the invention are expressed recombinantly in plant cells to enhance and increase levels of TZP polypeptides. Alternatively, antisense or other TZP constructs are used to suppress TZP levels of expression. A variety of different expression constructs, such as expression cassettes and vectors suitable for transformation of plant cells can be prepared. Techniques for transforming a wide variety of higher plant species are well known and described in the technical and scientific literature. See, e.g., Weising et al. Ann. Rev. Genet. 22:421-477 (1988). A TZP sequence coding for a TZP polypeptide, e.g., a cDNA sequence encoding a full length protein, can be combined with cis-acting (promoter) and trans-acting (enhancer) transcriptional regulatory sequences to direct the timing, tissue type and levels of transcription in the intended tissues of the transformed plant. Translational control elements can also be used.

The invention provides an TZP nucleic acid operably linked to a promoter that, in some embodiments, is capable of driving the transcription of the TZP coding sequence in plants. The promoter can be, e.g., derived from plant or viral sources. The promoter can be, e.g., constitutively active, inducible, or tissue specific. In construction of recombinant expression cassettes, vectors, transgenics, of the invention, a different promoters can be chosen and employed to differentially direct gene expression, e.g., in some or all tissues of a plant or animal. Typically, as discussed above, desired promoters are identified by analyzing the 5′ sequences of a genomic clone corresponding to the TZP genes described here.

A. Constitutive Promoters

A promoter fragment can be employed that will direct expression of TZP nucleic acid in all transformed cells or tissues, e.g. as those of a regenerated plant. The term “constitutive regulatory element” means a regulatory element that confers a level of expression upon an operatively linked nucleic molecule that is relatively independent of the cell or tissue type in which the constitutive regulatory element is expressed. A constitutive regulatory element that is expressed in a plant generally is widely expressed in a large number of cell and tissue types. Promoters that drive expression continuously under physiological conditions are referred to as “constitutive” promoters and are active under most environmental conditions and states of development or cell differentiation.

A variety of constitutive regulatory elements useful for ectopic expression in a transgenic plant are well known in the art. The cauliflower mosaic virus 35S (CaMV 35S) promoter, for example, is a well-characterized constitutive regulatory element that produces a high level of expression in all plant tissues (Odell et al., Nature 313:810-812 (1985)). The CaMV 35S promoter can be particularly useful due to its activity in numerous diverse plant species (Benfey and Chua, Science 250:959-966 (1990); Futterer et al., Physiol. Plant 79:154 (1990); Odell et al., supra, 1985). A tandem 35S promoter, in which the intrinsic promoter element has been duplicated, confers higher expression levels in comparison to the unmodified 35S promoter (Kay et al., Science 236:1299 (1987)). Other useful constitutive regulatory elements include, for example, the cauliflower mosaic virus 19S promoter; the Figwort mosaic virus promoter; and the nopaline synthase (nos) gene promoter (Singer et al., Plant Mol. Biol. 14:433 (1990); An, Plant Physiol. 81:86 (1986)).

Additional constitutive regulatory elements including those for efficient expression in monocots also are known in the art, for example, the pEmu promoter and promoters based on the rice Actin-1 5′ region (Last et al., Theor. Appl. Genet. 81:581 (1991); Mcelroy et al., Mol. Gen. Genet. 231:150 (1991); Mcelroy et al., Plant Cell 2:163 (1990)). Chimeric regulatory elements, which combine elements from different genes, also can be useful for ectopically expressing a nucleic acid molecule encoding an TZP polynucleotide (Comai et al., Plant Mol. Biol. 15:373 (1990)).

Other examples of constitutive promoters include the 1′- or 2′-promoter derived from T-DNA of Agrobacterium tumefaciens (see, e.g., Mengiste (1997) supra; O'Grady (1995) Plant Mol. Biol. 29:99-108); actin promoters, such as the Arabidopsis actin gene promoter (see, e.g., Huang (1997) Plant Mol. Biol. 1997 33:125-139); alcohol dehydrogenase (Adh) gene promoters (see, e.g., Millar (1996) Plant Mol. Biol. 31:897-904); ACT11 from Arabidopsis (Huang et al. Plant Mol. Biol. 33:125-139 (1996)), Cat3 from Arabidopsis (GenBank No. U43147, Zhong et al., Mol. Gen. Genet. 251:196-203 (1996)), the gene encoding stearoyl-acyl carrier protein desaturase from Brassica napus (Genbank No. X74782, Solocombe et al. Plant Physiol. 104:1167-1176 (1994)), GPc1 from maize (GenBank No. X15596, Martinez et al. J. Mol. Biol 208:551-565 (1989)), Gpc2 from maize (GenBank No. U45855, Manjunath et al., Plant Mol. Biol. 33:97-112 (1997)), other transcription initiation regions from various plant genes known to those of skill. See also Holtorf Plant Mol. Biol. 29:637-646 (1995).

B. Inducible Promoters

Alternatively, a promoter may direct expression of the TZP nucleic acid of the invention under the influence of changing environmental conditions or developmental conditions. Examples of environmental conditions that may effect transcription by inducible promoters include anaerobic conditions, elevated temperature, drought, or the presence of light. Such promoters are referred to herein as “inducible” promoters. For example, the invention incorporates the drought-inducible promoter of maize (Busk (1997) supra); the cold, drought, and high salt inducible promoter from potato (Kirch (1997) Plant Mol. Biol. 33:897-909).

Alternatively, plant promoters which are inducible upon exposure to plant hormones, such as auxins, are used to express the nucleic acids of the invention. For example, the invention can use the auxin-response elements E1 promoter fragment (AuxREs) in the soybean (Glycine max L.) (Liu (1997) Plant Physiol. 115:397-407); the auxin-responsive Arabidopsis GST6 promoter (also responsive to salicylic acid and hydrogen peroxide) (Chen (1996) Plant J. 10: 955-966); the auxin-inducible parC promoter from tobacco (Sakai (1996) 37:906-913); a plant biotin response element (Streit (1997) Mol. Plant. Microbe Interact. 10:933-937); and, the promoter responsive to the stress hormone abscisic acid (Sheen (1996) Science 274:1900-1902).

Promoters that are inducible upon exposure to chemicals reagents applied to the plant, such as herbicides or antibiotics, can also be used to express the nucleic acids of the invention. For example, the maize In2-2 promoter, activated by benzenesulfonamide herbicide safeners, can be used (De Veylder (1997) Plant Cell Physiol. 38:568-577); application of different herbicide safeners induces distinct gene expression patterns, including expression in the root, hydathodes, and the shoot apical meristem. TZP coding sequence can also be under the control of, e.g., a tetracycline-inducible promoter, e.g., as described with transgenic tobacco plants containing the Avena sativa L. (oat) arginine decarboxylase gene (Masgrau (1997) Plant J. 11:465-473); or, a salicylic acid-responsive element (Stange (1997) Plant J. 11:1315-1324; Uknes et al., Plant Cell 5:159-169 (1993); Bi et al., Plant J. 8:235-245 (1995)).

Other inducible regulatory elements include but are not limited to copper-inducible regulatory elements (Mett et al., Proc. Natl. Acad. Sci. USA 90:4567-4571 (1993); Furst et al., Cell 55:705-717 (1988)); tetracycline and chlor-tetracycline-inducible regulatory elements (Gatz et al., Plant J. 2:397-404 (1992); Roder et al., Mol. Gen. Genet. 243:32-38 (1994); Gatz, Meth. Cell Biol. 50:411-424 (1995)); ecdysone inducible regulatory elements (Christopherson et al., Proc. Natl. Acad. Sci. USA 89:6314-6318 (1992); Kreutzweiser et al., Ecotoxicol. Environ. Safety 28:14-24 (1994)); heat shock inducible regulatory elements (Takahashi et al., Plant Physiol. 99:383-390 (1992); Yabe et al., Plant Cell Physiol. 35:1207-1219 (1994); Ueda et al., Mol. Gen. Genet. 250:533-539 (1996)); and lac operon elements, which are used in combination with a constitutively expressed lac repressor to confer, for example, IPTG-inducible expression (Wilde et al., EMBO J. 11:1251-1259 (1992)). An inducible regulatory element useful in the transgenic plants of the invention also can be, for example, a nitrate-inducible promoter derived from the spinach nitrite reductase gene (Back et al., Plant Mol. Biol. 17:9 (1991)) or a light-inducible promoter, such as that associated with the small subunit of RuBP carboxylase or the LHCP gene families (Feinbaum et al., Mol. Gen. Genet. 226:449 (1991); Lam and Chua, Science 248:471 (1990)).

C. Tissue-Specific Promoters

Alternatively, the plant promoter may direct expression of the polynucleotide of the invention in a specific tissue (tissue-specific promoters). Tissue specific promoters are transcriptional control elements that are only active in particular cells or tissues at specific times during plant development, such as in vegetative tissues or reproductive tissues.

Examples of tissue-specific promoters under developmental control include promoters that initiate transcription only (or primarily only) in certain tissues, such as vegetative tissues, e.g., roots or leaves, or reproductive tissues, such as fruit, ovules, seeds, pollen, pistols, flowers, or any embryonic tissue. Reproductive tissue-specific promoters may be, e.g., ovule-specific, embryo-specific, endosperm-specific, integument-specific, seed and seed coat-specific, pollen-specific, petal-specific, sepal-specific, or some combination thereof.

Other tissue-specific promoters include seed promoters. Suitable seed-specific promoters are derived from the following genes: MAC1 from maize (Sheridan (1996) Genetics 142:1009-1020); Cat3 from maize (GenBank No. L05934, Abler (1993) Plant Mol. Biol. 22:10131-1038); vivparous-1 from Arabidopsis (Genbank No. U93215); atmyc1 from Arabidopsis (Urao (1996) Plant Mol. Biol. 32:571-57; Conceicao (1994) Plant 5:493-505); napA from Brassica napus (GenBank No. J02798, Josefsson (1987) JBL 26:12196-1301); and the napin gene family from Brassica napus (Sjodahl (1995) Planta 197:264-271).

A variety of promoters specifically active in vegetative tissues, such as leaves, stems, roots and tubers, can also be used to express the TZP nucleic acids of the invention. For example, promoters controlling patatin, the major storage protein of the potato tuber, can be used, see, e.g., Kim (1994) Plant Mol. Biol. 26:603-615; Martin (1997) Plant J. 11:53-62. The ORF13 promoter from Agrobacterium rhizogenes which exhibits high activity in roots can also be used (Hansen (1997) Mol. Gen. Genet. 254:337-343. Other useful vegetative tissue-specific promoters include: the tarin promoter of the gene encoding a globulin from a major taro (Colocasia esculenta L. Schott) corm protein family, tarin (Bezerra (1995) Plant Mol. Biol. 28:137-144); the curculin promoter active during taro corm development (de Castro (1992) Plant Cell 4:1549-1559) and the promoter for the tobacco root-specific gene TobRB7, whose expression is localized to root meristem and immature central cylinder regions (Yamamoto (1991) Plant Cell 3:371-382).

Leaf-specific promoters, such as the ribulose biphosphate carboxylase (RBCS) promoters can be used. For example, the tomato RBCS1, RBCS2 and RBCS3A genes are expressed in leaves and light-grown seedlings, only RBCS1 and RBCS2 are expressed in developing tomato fruits (Meier (1997) FEBS Lett. 415:91-95). A ribulose bisphosphate carboxylase promoters expressed almost exclusively in mesophyll cells in leaf blades and leaf sheaths at high levels, described by Matsuoka (1994) Plant J. 6:311-319, can be used. Another leaf-specific promoter is the light harvesting chlorophyll a/b binding protein gene promoter, see, e.g., Shiina (1997) Plant Physiol. 115:477-483; Casal (1998) Plant Physiol. 116:1533-1538. The Arabidopsis thaliana myb-related gene promoter (Atmyb5) described by L1 (1996) FEBS Lett. 379:117-121, is leaf-specific. The Atmyb5 promoter is expressed in developing leaf trichomes, stipules, and epidermal cells on the margins of young rosette and cauline leaves, and in immature seeds. Atmyb5 mRNA appears between fertilization and the 16 cell stage of embryo development and persists beyond the heart stage. A leaf promoter identified in maize by Busk (1997) Plant J. 11:1285-1295, can also be used.

Another class of useful vegetative tissue-specific promoters are meristematic (root tip and shoot apex) promoters. For example, the “SHOOTMERISTEMLESS” and “SCARECROW” promoters, which are active in the developing shoot or root apical meristems and are described by Di Laurenzio (1996) Cell 86:423-433; and, Long (1996) Nature 379:66-69, can be used. Another promoter is the 3-hydroxy-3-methylglutaryl coenzyme A reductase HMG2 gene promoter, whose expression is restricted to meristematic and floral (secretory zone of the stigma, mature pollen grains, gynoecium vascular tissue, and fertilized ovules) tissues (see, e.g., Enjuto (1995) Plant Cell. 7:517-527). Additional promoter examples include the kn1-related gene promoters from maize and other species that show meristem-specific expression, see, e.g., Granger (1996) Plant Mol. Biol. 31:373-378; Kerstetter (1994) Plant Cell 6:1877-1887; Hake (1995) Philos. Trans. R. Soc. Lond. B. Biol. Sci. 350:45-51. One such example is the Arabidopsis thaliana KNAT1 promoter. In the shoot apex, KNAT1 transcript is localized primarily to the shoot apical meristem; the expression of KNAT1 in the shoot meristem decreases during the floral transition and is restricted to the cortex of the inflorescence stem (see, e.g., Lincoln (1994) Plant Cell 6:1859-1876).

One of skill will recognize that a tissue-specific promoter may drive expression of operably linked sequences in tissues other than the target tissue. Thus, as used herein a tissue-specific promoter is one that drives expression preferentially in the target tissue, but may also lead to some expression in other tissues as well.

In another embodiment, a TZP nucleic acid is expressed through a transposable element. This allows for constitutive, yet periodic and infrequent expression of the constitutively active polypeptide. The invention also provides for use of tissue-specific promoters derived from viruses which can include, e.g., the tobamovirus subgenomic promoter (Kumagai (1995) Proc. Natl. Acad. Sci. USA 92:1679-1683; the rice tungro bacilliform virus (RTBV), which replicates only in phloem cells in infected rice plants, with its promoter which drives strong phloem-specific reporter gene expression; the cassaya vein mosaic virus (CVMV) promoter, with highest activity in vascular elements, in leaf mesophyll cells, and in root tips (Verdaguer (1996) Plant Mol. Biol. 31:1129-1139).

### V. Production of Transgenic Plants

DNA constructs of the invention may be introduced into the genome of the desired plant host by a variety of conventional techniques. For example, the DNA construct may be introduced directly into the genomic DNA of the plant cell using techniques such as electroporation and microinjection of plant cell protoplasts, or the DNA constructs can be introduced directly to plant tissue using ballistic methods, such as DNA particle bombardment. Alternatively, the DNA constructs may be combined with suitable T-DNA flanking regions and introduced into a conventional Agrobacterium tumefaciens host vector. The virulence functions of the Agrobacterium tumefaciens host will direct the insertion of the construct and adjacent marker into the plant cell DNA when the cell is infected by the bacteria.

Microinjection techniques are known in the art and well described in the scientific and patent literature. The introduction of DNA constructs using polyethylene glycol precipitation is described in Paszkowski et al. EMBO J. 3:2717-2722 (1984). Electroporation techniques are described in Fromm et al. Proc. Natl. Acad. Sci. USA 82:5824 (1985). Ballistic transformation techniques are described in Klein et al. Nature 327:70-73 (1987).

Agrobacterium tumefaciens-mediated transformation techniques, including disarming and use of binary vectors, are well described in the scientific literature. See, for example Horsch et al. Science 233:496-498 (1984), and Fraley et al. Proc. Natl. Acad. Sci. USA 80:4803 (1983).

Transformed plant cells that are derived from any transformation technique can be cultured to regenerate a whole plant that possesses the transformed genotype and thus the desired phenotype. Such regeneration techniques rely on manipulation of certain phytohormones in a tissue culture growth medium, optionally relying on a biocide and/or herbicide marker that has been introduced together with the desired nucleotide sequences. Plant regeneration from cultured protoplasts is described in Evans et al., Protoplasts Isolation and Culture, Handbook of Plant Cell Culture, pp. 124-176, MacMillilan Publishing Company, New York, 1983; and Binding, Regeneration of Plants, Plant Protoplasts, pp. 21-73, CRC Press, Boca Raton, 1985. Regeneration can also be obtained from plant callus, explants, organs, or parts thereof. Such regeneration techniques are described generally in Klee et al. Ann. Rev. of Plant Phys. 38:467-486 (1987).

The nucleic acids of the invention can be used to confer desired traits on essentially any plant. Thus, the invention has use over a broad range of plants, including species from the genera Asparagus, Atropa, Avena, Brassica, Citrus, Citrullus, Capsicum, Cucumis, Cucurbita, Daucus, Fragaria, Glycine, Gossypium, Helianthus, Heterocallis, Hordeum, Hyoscyamus, Lactuca, Linum, Lolium, Lycopersicon, Malus, Manihot, Majorana, Medicago, Nicotiana, Oryza, Panieum, Pannesetum, Persea, Pisum, Pyrus, Prunus, Raphanus, Secale, Senecio, Sinapis, Solanum, Sorghum, Trigonella, Triticum, Vitis, Vigna, and, Zea.

### EXAMPLES

The following examples are offered to illustrate, but not to limit the claimed invention.

In this example, we utilize the hypocotyl assay to identify QTL controlling growth in two light and two temperature conditions. We identified a recessive large effect QTL on chromosome five controlling 40% of the growth variation segregating in Recombinant Inbred Lines (RILs) derived from the Bay-0 and Shandara accessions of A. thaliana. The QTL was fine-mapped and the causal factor shown to be a mutation affecting a tandem zinc knuckle/PLUS3 protein (At5g43630; TZP), which is encoded by a single-copy gene in all completely sequenced plant genomes. We show that TZP acts downstream of the circadian clock and light signaling, directly regulating blue light-dependent, morning (dawn) specific growth, during seedling development and beyond. Based on its nuclear localization and its novel domain structure, we argue that TZP functions at the transcriptional level to control growth-promoting pathways. TZP represents a new component of the growth pathway that was not previously identified using traditional genetic screens.

### Mapping QTL for Hypocotyl Elongation in the Bay-0×Shandara RIL Population

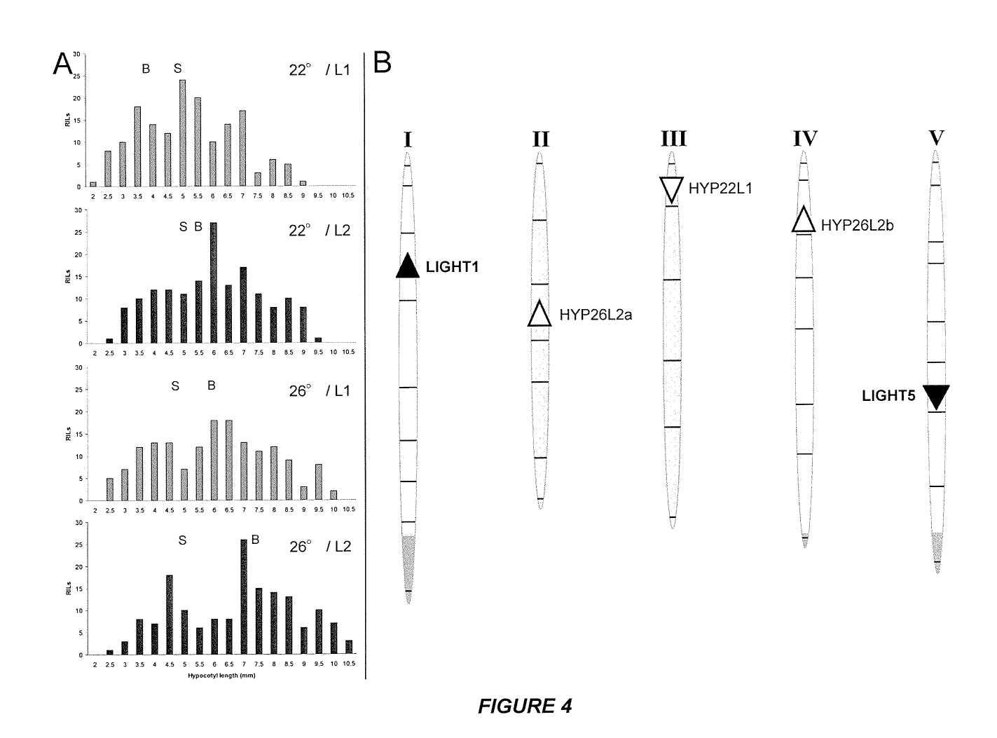

Intraspecific variation provides a fertile source of genetic combinations that can be utilized to map new genes (or new alleles) involved in complex traits such as growth. To investigate natural variation for hypocotyl elongation response to light and temperature, we phenotyped a core set of 164 RILs from the Bay-0×Shandara cross in four different environments combining two white-light (17 mmol/m⁻²s⁻¹ μL1] and 10 μmol/m⁻²s⁻¹ [L2]) and two temperature (22° C. and 26° C.) conditions (supporting information [SI] FIG. 4A). The parental phenotypes reveal that in our conditions Shandara responds poorly to temperatures above 22° C. or light below 17 μmol·m⁻²·s⁻¹, or a combination of both. In contrast, Bay-0 responds strongly to both temperature and light, with a synergistic interaction between both factors. Variation among the RILs seems to follow parental variation with signs of bimodality (especially at 26° C./L1 and 26° C./L2) suggesting the segregation of some large-effect QTL. Transgression was also prevalent in all conditions and in both directions. Overall, genotypic variation was significant in each environment and broad-sense heritability of the trait was accordingly high, above 70% (see table S1 from our website data at www.inra.fr/vast/Files/Loudet_PNAS_SITables.xls). While RIL response to contrasted temperature and light treatments was significant (P<0.001), RIL×light interactions were not significant at either 22° C. or 26° C., and the RIL×temperature interaction was only significant under L1 (data not shown). This indicates that most of the phenotypic variation between RILs is stable and conserved across environments.

The genetic architecture of variation in hypocotyl elongation under these environmental conditions is presented in SI FIG. 4 B (see also table S1 from our website data). Two loci with major effects are detected across all environments and called LIGHT1 and LIGHT5, whereas the remaining loci called ‘HYP’ are specific to a single environment with more subtle phenotypic contributions (only 4% each). The identification of these two major effect QTL is in accordance with the meager RIL×environment interactions found. The LIGHT5 locus explains over 40% of the variance, with no LIGHT5×environment interaction found in any condition (data not shown). Its negative allelic effect is predicted to represent a combined 2.1 to 2.5 mm increase in hypocotyl elongation contributed by Shandara alleles (Sha) relative to the Bay-0 alleles (Bay). In contrast, Sha alleles at LIGHT1 are responsible for a decrease in hypocotyl elongation compared to Bay alleles, but with a relatively smaller phenotypic contribution (explaining 25 to 30% of the variance). The opposite allelic effects of LIGHT1 and LIGHT5 are responsible for most of the transgression observed in each environment. LIGHT1 interacts with temperature under either L1 or L2 conditions and with light at either 22° C. or 26° C. (data not shown). There is no significant epistatic relationship between LIGHT1 and LIGHT5, or among any other pair of loci.

### Confirmation and Fine-Mapping of Lights to Three Candidate Genes

Identifying the Quantitative Trait Gene (QTG) underlying a QTL is a challenging task that requires several independent lines of proof that a gene is linked to a trait of interest and that variation in this gene explains the trait (Weigel, D. and Nordborg, M., Plant Physiol, 138:567-568 (2005)). The most general approach is fine-mapping to a very small physical candidate interval, which in the best case allows immediate identification of candidate polymorphisms (Quantitative Trait Nucleotide, QTN) within the causal gene or regulatory regions (Clark, R. M. et al., Science, 317:338-342 (2007)). For most QTL and situations in A. thaliana, phenotyping for a QTL effect remains much more limiting than genotyping many individuals. Therefore, an efficient strategy for fine-mapping is to first isolate recombinants within a segregating nearly-isogenic line based on genotype alone and then to phenotypically interrogate only the informative ones (in successive rounds) to reduce the candidate interval to the gene level (Peleman, J. D. et al., Genetics, 171:1341-1352 (2005)).

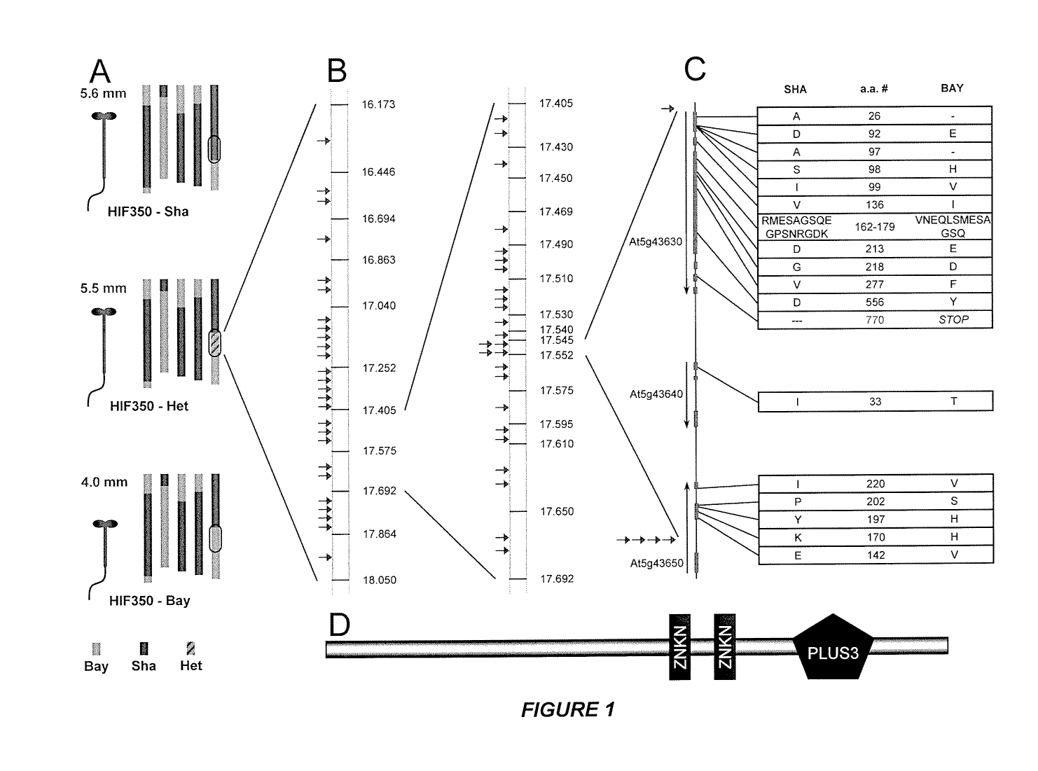

We followed the HIF strategy to build nearly-isogenic lines from a RIL (RIL350) that was segregating solely for the LIGHT5 region. Comparing plants homozygous for the Sha allele with plants homozygous for the Bay allele at the QTL region (in an otherwise identical genetic background) confirmed the phenotypic impact of LIGHT5 on hypocotyl elongation (FIG. 1A). HIF350-Sha hypocotyls are consistently 1.6 mm longer than those of HIF350-Bay (slightly less than predicted by the QTL analysis). The analysis of heterozygous plants showed that the Sha allele of LIGHT5 is fully dominant over the Bay allele (FIG. 1A). The phenotypic effect observed was identical when first fixing alternate genotypes at the QTL region and then comparing the phenotypes of the descendants produced by those homozygous plants (“fixed progeny”) or when directly studying the segregating descendants of a heterozygous plant (“progeny testing”). This precludes any maternal phenotypic effect and demonstrates a direct control of the phenotype expressed in seedlings by the LIGHT5 alleles.

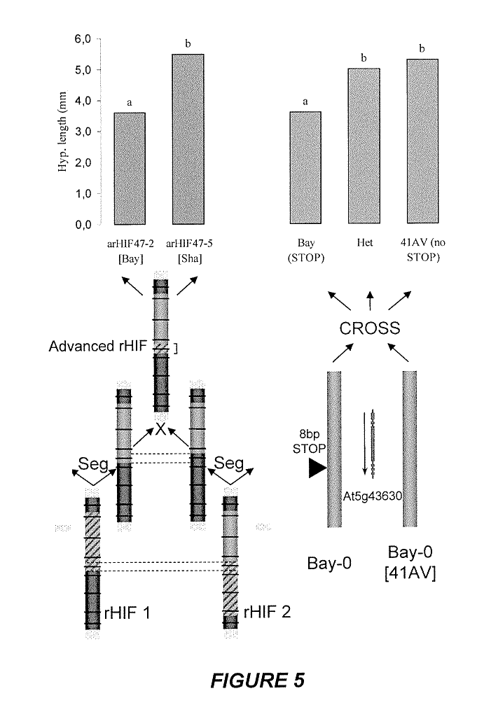

Screening for recombinants by genotyping 600 plants descended from the initial RIL350 individual (heterozygous over the whole QTL region) allowed us to identify 80 recombinants (recombined HIF, or rHIF) over the ˜1.9 Mb heterozygous region. Twenty-six of these rHIF were individually interrogated in successive rounds of progeny testing to score for the presence (or absence) of the QTL effect caused by the remaining interval. This first screen allowed us to narrow the candidate region to less than 300 kb between markers at 17.405 and 17.692 Mb (FIG. 1B). Screening 4,000 descendants from one of the positive rHIFs found in the previous step allowed us to identify 73 new rHIFs within the 300 kb candidate interval; 25 of these were individually progeny tested to define a smaller interval containing the QTL (FIG. 1B). Results were consistent among rHIFs, and the last five recombinants delimited a 7 kb interval, from 17.545 Mb (one recombinant) to 17.552 Mb (four independent recombinants; FIG. 1C). Three predicted genes, At5g43630, At5g43640, and At5g43650, are at least partially included in the 7 kb candidate region. We crossed two independent rHIFs with appropriate genotypes (see SI FIG. 5) in order to generate a line that was segregating only for the candidate region (and fixed to the north and south), following a strategy suggested by Kroymann and Mitchell-Olds (Kroymann, J. and Mitchell-Olds, T., Nature, 435:95-98 (2005)) that we named ‘advanced rHIF (arHIF) cross’. This approach confirmed that the 17.545-17.552 Mb interval was sufficient to recapitulate the LIGHT5 phenotype (SI FIG. 5).

Sequencing the 7 kb interval in Bay-0 and Shandara revealed dozens of SNPs and several indels, many of which resulted in non-silent changes in the coding regions of the three candidate genes. Eight, one and five SNPs caused amino-acid changed in At5g43630, At5g43640 and At5g43650, respectively. Two single amino-acid deletions and one larger deletion were discovered in At5g43630. Finally, an 8 by insertion in Bay-0 caused a frameshift and a premature stop within the coding region for the predicted PLUS3 domain of At5g43630 (FIGS. 1C and D).

### Identification of the LIGHT5 Causal Gene as Tandem Zinc Knuckle/Plus3 (TZP)

The three genes in the LIGHT5 interval are annotated as encoding a tandem zinc knuckle/PLUS3 (TZP, At5g43630), a 40S ribosomal protein (RPS15E, At5g43640), and a basic helix-loop-helix protein (bHLH092, At5g43650). TZP has no close homologs, but does share similarity with other Arabidopsis proteins that only have the zinc knuckle or PLUS3 domains. bHLH092 is part of the large bHLH family of transcription factors (Toledo-Ortiz, G. et al., Plant Cell, 15:1749-1770 (2003)), and a close homolog is TRANSPARENT TESTA 8 (TT8; At4g09820). There are four closely related RPS15E genes (At1g04270, At5g09500, At5g09510, and At5g09490) (Barakat, A. et al., Plant Physiol, 127:398-415 (2001)). The transcript/expression of all three annotated genes in the Columbia background was confirmed by tiling array data and full length cDNAs. Quantitative RT-PCR (qPCR) revealed that all three genes are expressed in Bay-0 and Shandara (data not shown).

T-DNA-insertions in RPS15E or bHLH092 did not result in hypocotyl elongation phenotypes (data not shown). No insertion mutants were available for TZP (an insertion GABI-KAT line could not be recovered). To determine the association of the stop codon in TZP with the hypocotyl phenotype, we sequenced the PLUS3 domain in other A. thaliana accessions. Although we identified 10 synonymous changes and 13 non-synonymous changes (including two changes affecting residues at least partially conserved in other species), we could not detect the Bay-0 premature stop codon polymorphism in a panel of ˜300 additional accessions (data not shown). We discovered that a Bay-0 single seed descent (SSD) line (Bay-0[41AV]), which was derived independently of the parent for the RIL set, did not have the 8 bp-insertion in TZP. Sequencing the entire 7 kb-candidate region revealed that this was the only sequence difference between Bay-0 and Bay-0[41AV] at LIGHT5. Genotyping additional markers in Bay-0[41AV] (as well as a careful phenotypic observation of the lines) showed that it is from the same genetic background as Bay-0 (data not shown). We crossed the two lines, with and without the TZP stop codon, to determine whether the mutation was responsible for the LIGHT5 phenotype. As shown in SI FIG. 5, phenotypes of F2 plants homozygous for either allele were very similar to the phenotypes of the arHIF fixed for either the Bay (stop) or Sha allele, respectively. This demonstrates that the 8 bp-insertion in TZP is sufficient to explain the LIGHT5 phenotype, and allows us to conclude that TZP controls hypocotyl growth. Bay-0[41AV] is the only Bay-0 stock we have which does not carry the 8 bp-insertion. The question remains as to when this causative polymorphism appeared in the Bay-0 lineage (before or after collection) and whether it really exists in nature. Unfortunately, indications about the exact collection site of Bay-0 are very poor and make it nearly impossible to locate the original natural population and answer this question.

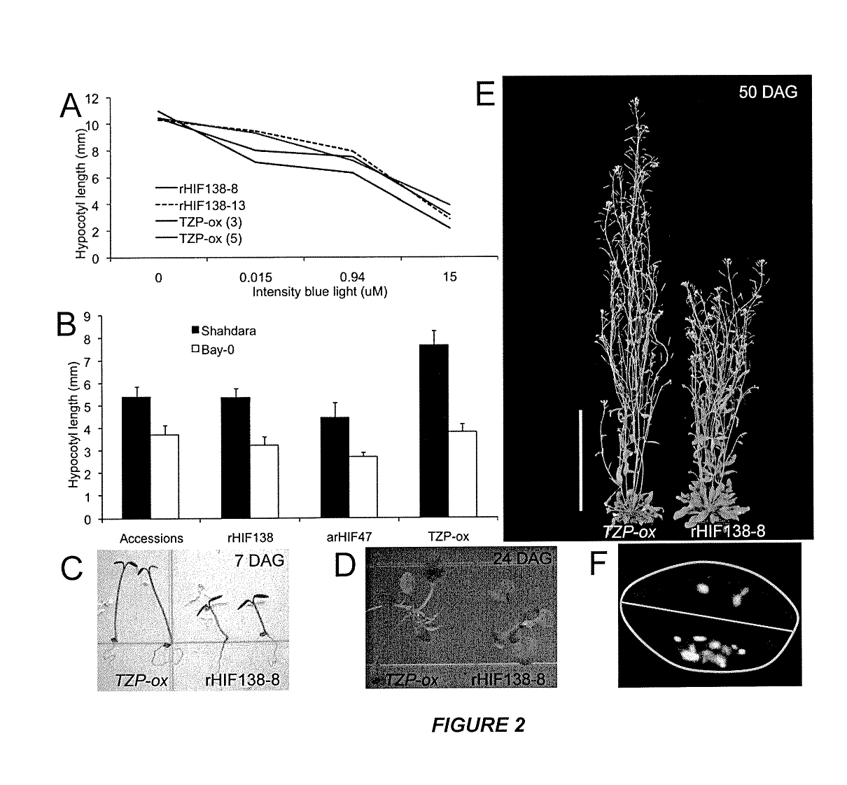

Additionally, we employed a transgenic approach to confirm the role of TZP in hypocotyl growth. The Sha alleles of all three genes were overexpressed in the rHIF containing the Bay allele (rHIF138-8). Overexpression of TZP (TZP-OX) caused plants to have very long hypocotyls (FIGS. 2B-C), with increased growth throughout development and extended duration of the reproductive phase (FIGS. 2D-E). All linearly-elongating organs were more extended, including petioles, internodes and peduncles and the main floral stem of TZP-OX plants was usually twice as long as in its background. In contrast, RPS15E or bHLH092 over-expressing plants were indistinguishable from the parental line in terms of growth (data not shown). Over-expression of bHLH092 resulted in white seeds, similar to a phenotype found in mutants of its closest homolog TT8 (Nesi, N. et al., Plant Cell, 12:1863-1878 (2000)). From these data we conclude that TZP is the QTG explaining the LIGHT5 QTL, and that the 8 by indel leading to a premature stop codon in the Bay-0 allele is the causative polymorphism.

### Tzp is a Large, Nuclear-Localized Protein Encoded by a Single Copy Gene

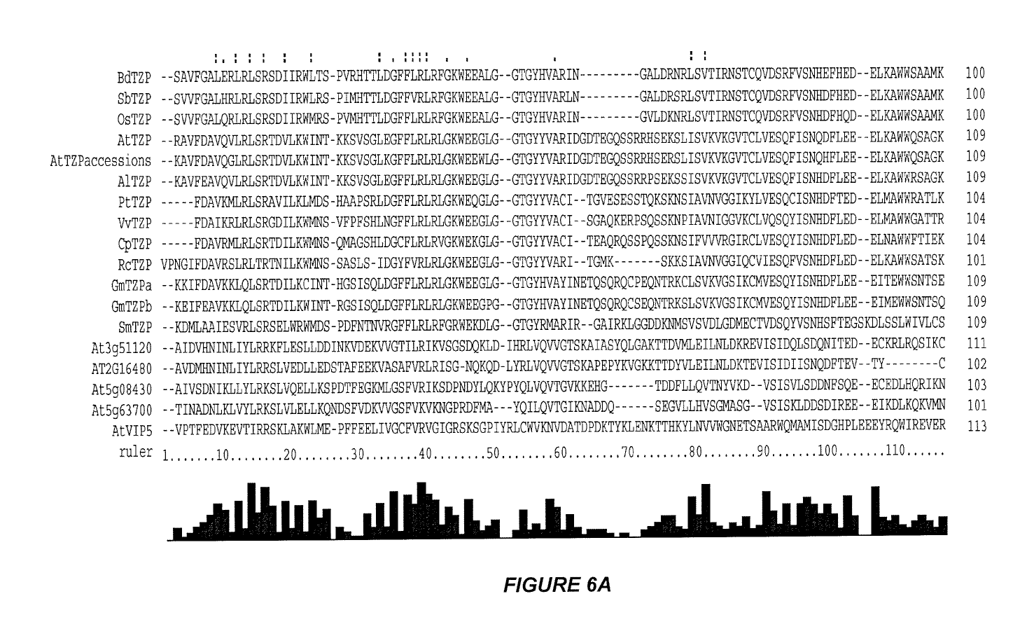

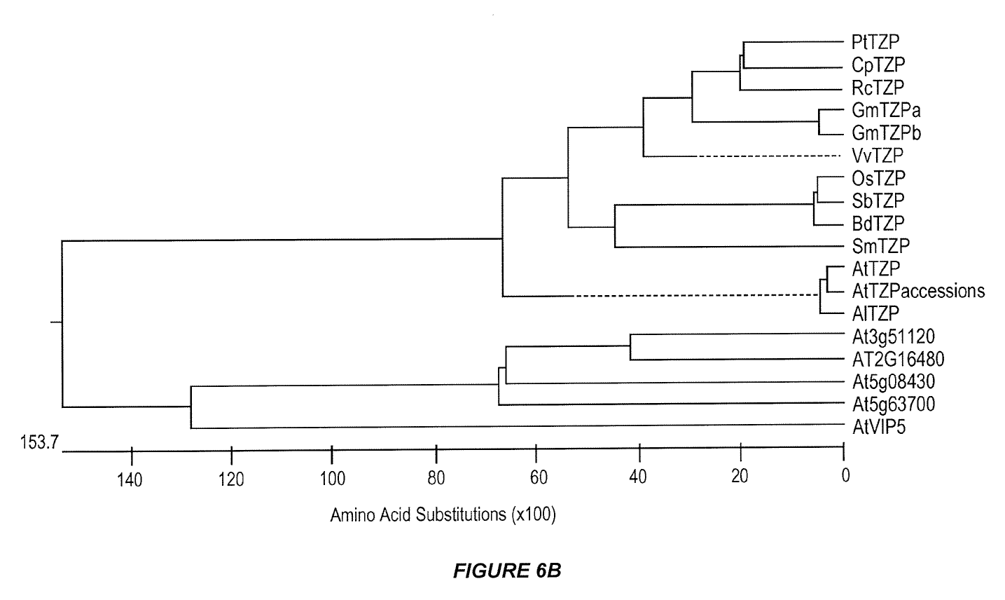

TZP is a single copy gene in A. thaliana. TZP orthologs are also single copy in the fully sequenced plant genomes (SI FIG. 6). Interestingly, TZP is not found in the moss Physcomitrella patens, or in Chlamydomonas reinhardtii or any earlier algal lineages queried, suggesting that it is specific to vascular plants.

The zinc knuckle (znkn; CX₂CX₄HX₄C; SEQ ID NO:13) domain has been shown to be important for protein-protein interactions, as well as for binding single-stranded DNA (Vo, L. T. et al., Mol Cell Biol, 21:8346-8356 (2001)). There are at least 24 znkn-containing proteins in Arabidopsis, five of which are closely related to each other, but not to TZP (table S2 from our website data). Mutations in one of them, SWELLMAP 1 (SMP1), result in small plants with small cells because of a dysfunction in the commitment to the cell cycle (Clay, N. K. and Nelson, T., Plant Cell, 17: 1994-2008 (2005)). Another znkn protein, RSZ33, regulates interactions between splicing factors (Kalyna, M. et al., Mol Biol Cell, 14:3565-3577 (2003); Lopato, S. et al., Plant Mol Biol, 39:761-773 (1999)). In yeast, the znkn of MPE1 has been found to control 3′ end processing of pre-mRNA, and its human ortholog RBBP6 has been shown to interact with tumor suppressor pRB1 (Vo, L. T. et al., Mol Cell Biol, 21:8346-8356 (2001)). Znkn domains are also found in retroviral gag proteins (nucleocapsid), including that of HIV (De Guzman, R. N. et al., Science, 279:384-388 (1998)).

The PLUS3 domain is thought to play a role in nucleic acid binding through three conserved positive amino acids (de Jong, R. N. et al., Structure, 16:149-159 (2008)). There are five proteins in Arabidopsis with the PLUS3 domain (SI FIG. 6 and our website data). Three of these also have a SWIB domain, two have either a CCCH or a C3H3C4 zinc finger, and VERNALIZATION INDEPENDENT 5 (VIPS) contains only the PLUS3 domain (Oh, S. et al., Plant Cell, 16:2940-2953 (2004)). VIPS is a homolog of the yeast RTF1 protein, which is part of the yeast Paf1 complex that regulates histone H₂B ubiquitination, histone H3 methylation, RNA polymerase II carboxy-terminal Ser2 phosphorylation and RNA 3′ end processing (de Jong, R. N. et al., Structure, 16:149-159 (2008)). The SWIB domain-containing proteins are part of the SWI/SNF chromatin-remodeling complex (Mlynarova, L. et al., Plant J, 51:874-885 (2007)). The combination of tandem zinc knuckles and a PLUS3 domain is unique to TZP-type genes in plants. Based on GFP fusions, TZP is localized to the nucleus in small punctate structures (FIG. 2F). Considering the domain structure and the localization, it seems most likely that TZP has a role in transcriptional control, perhaps at the level of chromatin remodeling.

### TZP Regulates Light Quality-Dependent Growth

Our initial QTL study suggested that LIGHT5 is involved in light-regulated hypocotyl growth. To determine whether the growth defects in LIGHT5 are specific to certain light environments, we measured hypocotyl lengths for both the rHIF and TZP-OX lines under different fluences of monochromatic red, blue and far-red light, and in continuous dark. We found that both loss and gain of TZP activity had a significant effect on hypocotyl length under a range of blue or white fluences, but not in red or far-red light, or in the dark (FIGS. 2A-B; data not shown). These results are consistent with TZP playing a role in light-dependent hypocotyl elongation, and suggest that TZP is involved in blue-light signaling.

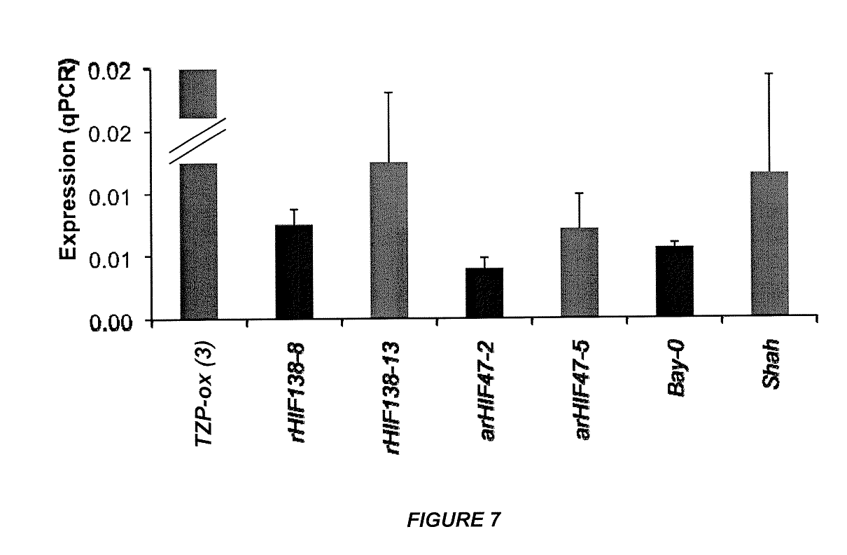

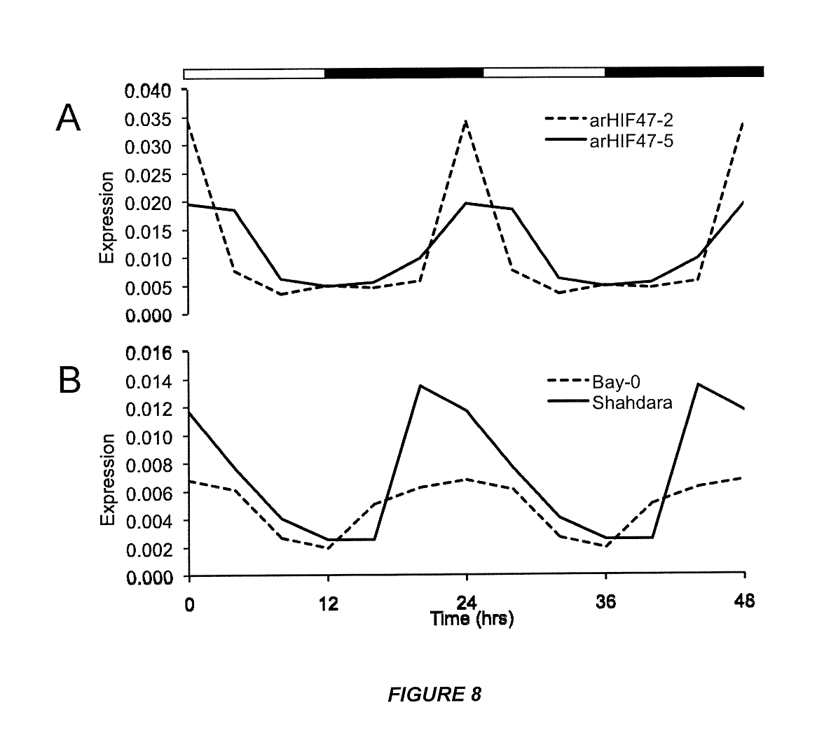

We measured transcript abundance in Shandara, Bay-0, rHIF138-8, rHIF138-13, arHIF47-2, arHIF47-5, and TZP-OX line #3 seedlings grown in constant blue light (15 μmol/m²s) for five days and then harvested at subjective dawn (relative to the time when they were moved from stratification to light). The extent of growth paralleled TZP expression levels (SI FIG. 7), consistent with TZP having a direct effect on growth. To identify potential downstream transcriptional targets of TZP, we analyzed genome-wide expression patterns of five genotypes (rHIF138-8 and rHIF138-13; arHIF47-2 and arHIF47-5; TZP-OX line#3) using Affymetrix Arabidopsis ATH1 GeneChip arrays. In pair-wise comparisons, we found 135, 12 and 769 genes to be differentially expressed between lines with contrasting alleles, rHIF138-8 vs. rHIF138-13, arHIF47-2 vs. arHIF47-5, and TZP-OX vs. rHIF138-8, respectively (P<0.01; tables S3-S6 from our website data; Materials and Methods in SI Text). The top four significant gene ontology categories in the comparison of TZP-OX and rHIF138-8 (769 genes) are cytosol, ribosome, structural molecule activity, and cell wall (table S7 from our website data), consistent with TZP playing a specific role in modulating growth. We found similar results with the rHIF138-8 vs. rHIF138-13 comparison (data not shown).

### TZP Controls Morning-Specific Growth Through an Auxin-Related Pathway

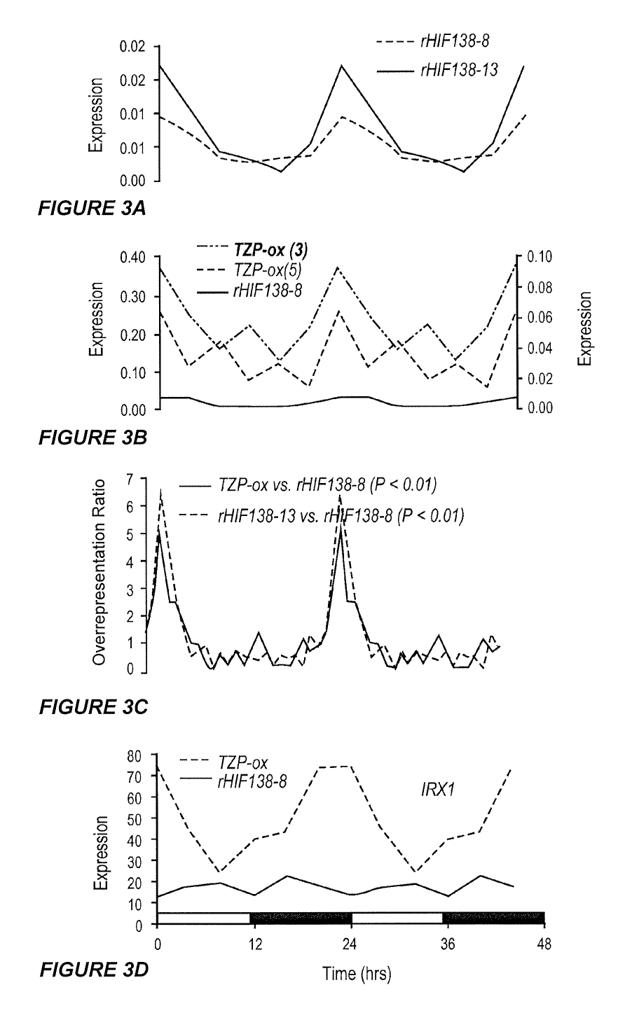

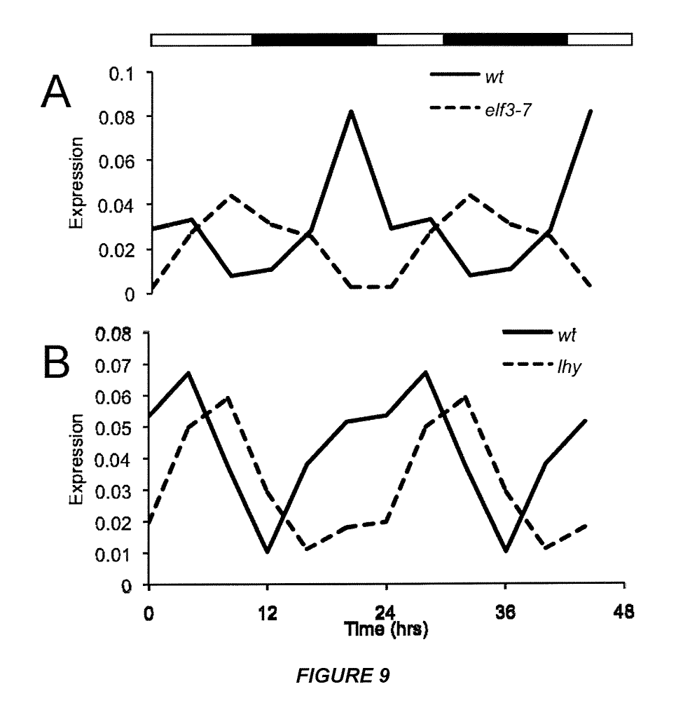

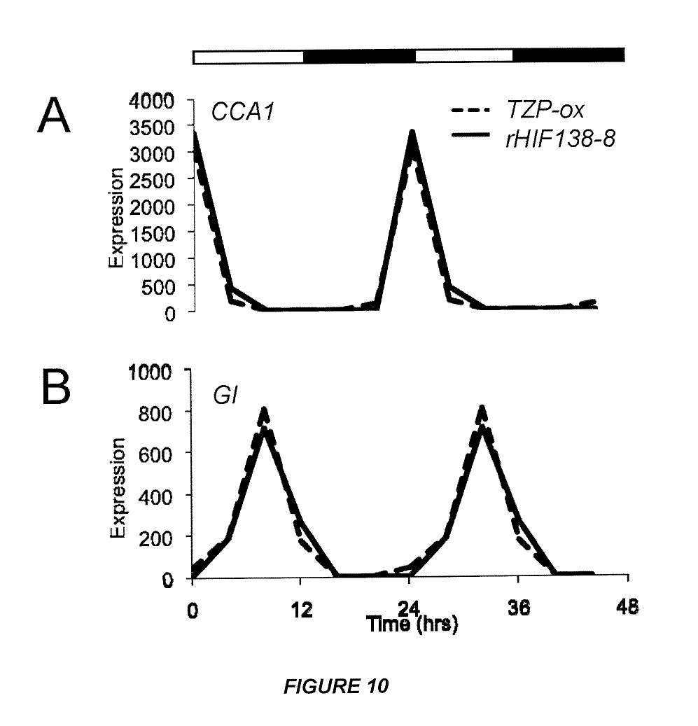

It is well established that hypocotyl elongation is controlled by the circadian clock (Nozue, K. et al., Nature, 448:358-361 (2007); Dowson-Day, M. J. and Millar, A. J., Plant J, 17:63-71 (1999)). Therefore, we tested whether TZP is clock regulated and if the circadian clock is altered in TZP-OX plants and the rHIF carrying the Bay-0 allele. We found that TZP cycles under both diurnal and circadian conditions with peak expression at dawn (transition from dark to light; FIG. 3A; SI FIG. 8). Hypocotyl growth is maximal at dawn and many genes that regulate growth, such as cell wall and phytohormone genes, have dawn-specific transcript abundance (Nozue, K. et al., Nature, 448:358-361 (2007); Michael, T. P. et al., PLoS Biology, (in press) (2008)). The dawn-specific TZP transcript peak suggests that it may be part of a circadian-controlled growth mechanism. To confirm that the clock controls TZP, we asked if circadian mutants disrupt expression of TZP. Indeed, TZP expression was changed in both early flowering3 (elf3) and late elongated hypocotyl (lhy) mutants (SI FIG. 9). However, the circadian clock is not disrupted in either the rHIF or TZP-OX under light/dark cycles (SI FIG. 10), consistent with no feedback of TZP into the circadian clock. Based on these results we propose that TZP functions to control growth downstream of the circadian clock.

Since TZP is regulated by light/dark cycles and the circadian clock, we asked if the genes disrupted by TZP-OX were time-of-day specific. We used the PHASER time-of-day analysis tool (http://phaser.cgrb.oregonstate.edu/), which determines if there is a pattern of time-of-day co-expression in a given gene list compared to a background model. The peak expression of genes that were disrupted in TZP-OX, and differentially expressed in rHIF138-8 vs. rHIF138-13 is biased towards dawn (FIG. 3C). This is consistent with the dawn-specific expression of TZP. In addition, morning-specific response elements such as the morning element (CCACA), Gbox (CACGTG) and HUD (CACATG) [table S8 from our website data; (Michael, T. P. et al., PLoS Biology, (in press) (2008); Michael, T. P. et al., PLoS Genet, 4:e14 (2008))] were overrepresented in the promoters (500 bp) of these genes as determined using the ELEMENT motif-searching tool (Mockler, T. C. et al., Cold Spring Harb Symp Quant Biol, 72:353-363 (2007)); http://element.cgrb.oregonstate.edu/). These results support a role of TZP in the transcriptional activity of dawn-specific genes.

In an effort to capture the entire effect of TZP-OX, we carried out a time course under light/dark cycles (12 hrs white light/12 hrs dark) in seven-day-old seedlings, sampling every four hours over one day in the same genotypes as above. We validated the overexpression of TZP using qPCR, which revealed that TZP continued to cycle despite overexpression (FIG. 3B), suggesting that TZP is also controlled post-transcriptionally. Global expression changes were assessed using Affymetrix ATH1 GeneChip arrays in TZP-OX, rHIF138-8 and rHIF138-13. The resulting time courses were analyzed for differentially expressed genes and for cycling genes [(Michael, T. P. et al., PLoS Genet, 4:e14 (2008)); tables S9-S12 from our website data; Materials and Methods in SI text]. Using the time points as replicates, we identified 117 upregulated and 40 downregulated genes in TZP-OX (P<0.01).

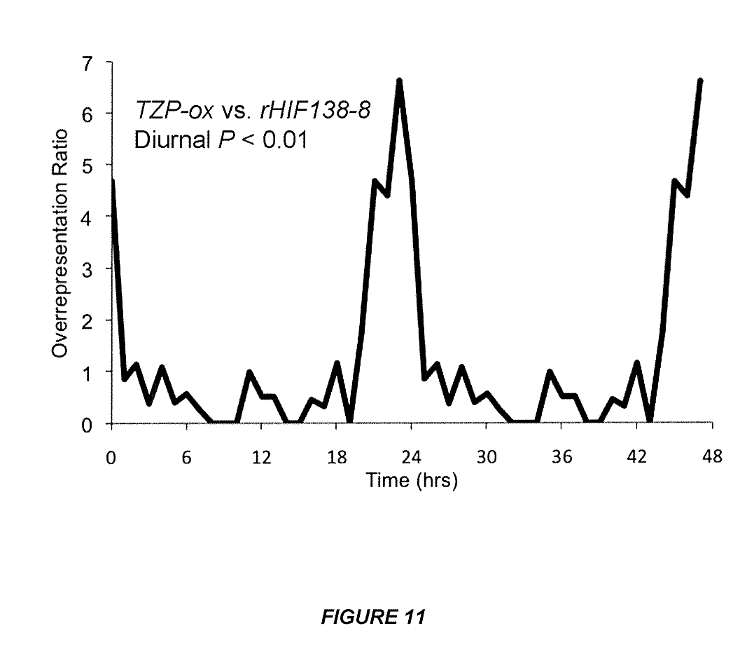

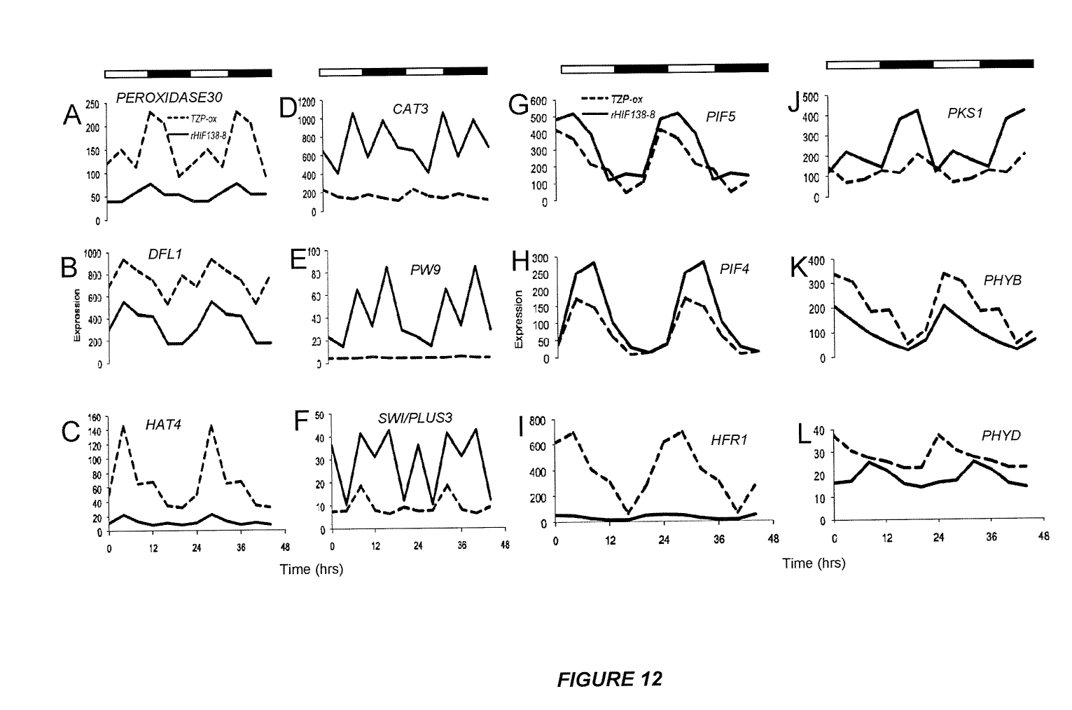

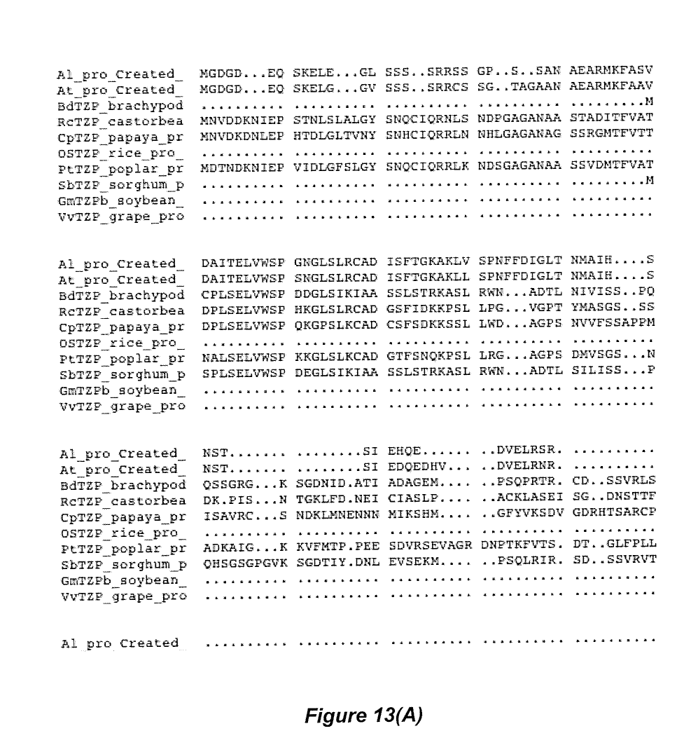

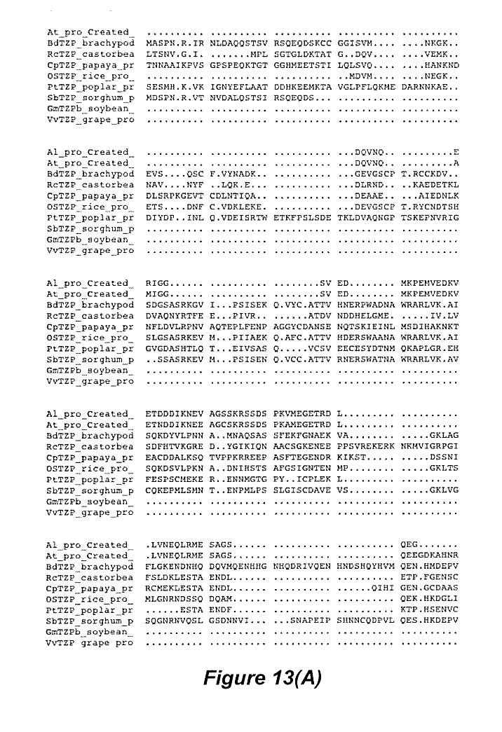

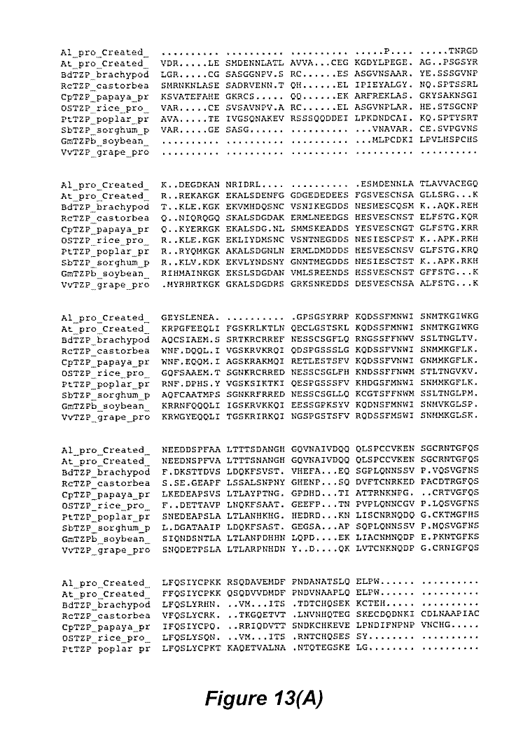

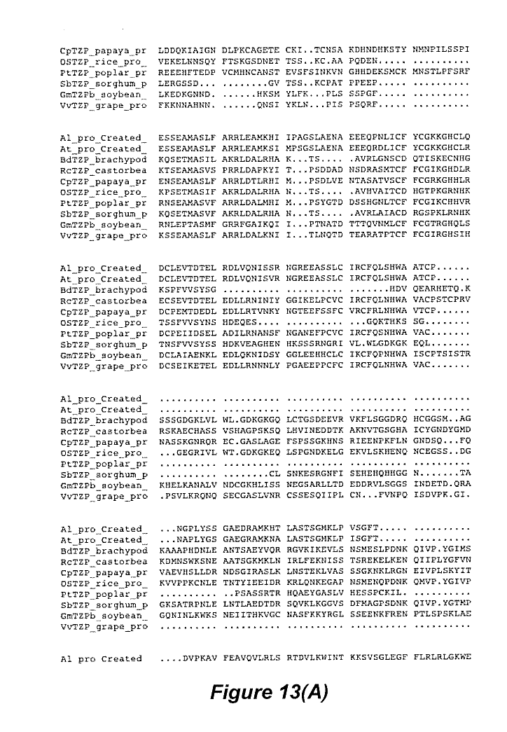

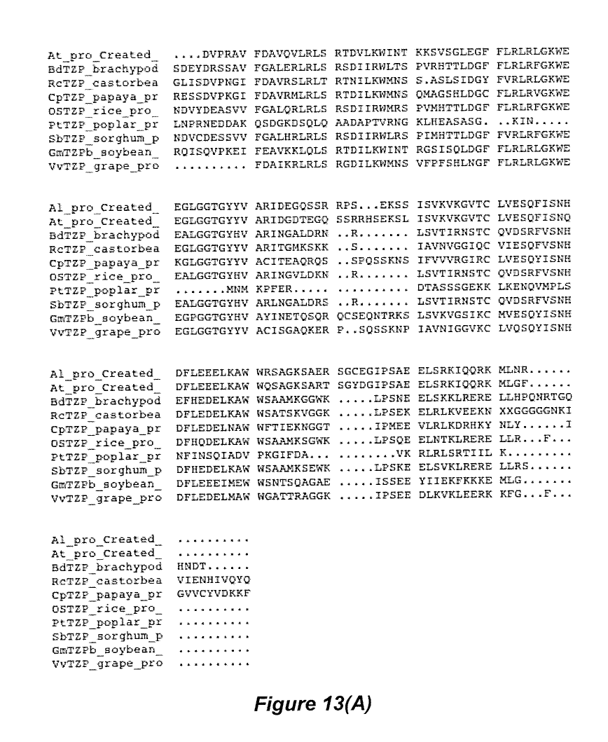

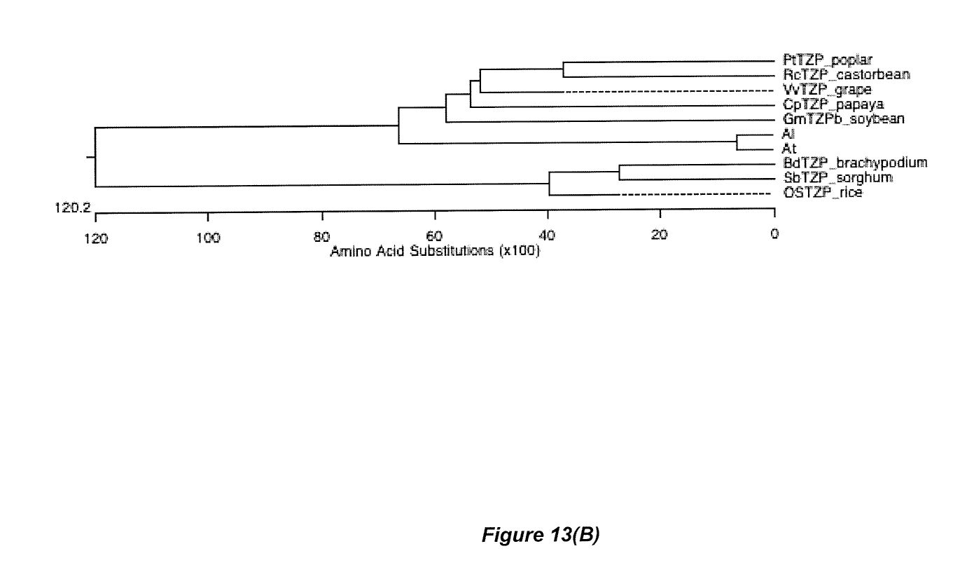

Similar to the results in blue light, the genes that were upregulated in TZP-OX were dawn-specific (SI FIG. 11) and cell wall genes were overrepresented (table S13 from our website data). IRX1 is one example of the eight cell wall genes that were upregulated in TZP-OX (FIG. 3D). Like IRX1, many of the upregulated genes were specifically overexpressed at dawn, similar to the overexpression pattern of TZP itself (FIG. 3A; SI FIG. 12). Peroxidases, which can function to polymerize cell wall compounds, were also upregulated in TZP-OX, consistent with their role in growth and cell wall expansion (Passardi, F. et al., Planta, 223:965-974 (2006)). Peroxidases PER27, PER30, and PER64 have been shown to be part of the cell wall proteome (Bayer, E. M. et al., Proteomics, 6:301-311 (2006)) (6 PER genes total; SI FIG. 12A). However, CATALASE 3 (CAT3; SEN2) was one of the most downregulated genes (SI FIG. 12D). CAT3 cycles under all diurnal and circadian conditions and may play a role in senescence and stress responses (Zimmermann, P. et al., Plant Cell Environ, 29:1049-1060 (2006); Michael, T. P. and McClung, C. R., Plant Physiol, 130:627-638 (2002)). Two additional genes of note were downregulated in TZP-OX: PW9 (encoding a MATH/TRAF domain protein) and one of the PLUS3 homologs that encodes also a SWI/SNF domain (SI FIGS. 12E-F). In general, the genes that were misregulated in TZP-OX are involved in cell expansion, consistent with TZP being intimately related to regulation of hypocotyl elongation and downstream of the circadian clock.

Auxin-response genes, including WES™ [GH3.5; (Park, J. E. et al., Plant Cell Physiol, 48:1236-1241 (2007))], DFL1 [GH3.6; (Nakazawa, M. et al., Plant J, 25:213-221 (2001))] (SI FIG. 12B), and AXR5 [IAA1; (Yang, X. et al., Plant J, 40:772-782 (2004))], all of which have been shown to control hypocotyl growth, were also upregulated upon TZP overexpression. It has been shown recently that auxin controls growth in a time-of-day fashion (Covington, M. F. and Harmer, S. L., PLoS Biol, 5:e222 (2007)), which fits with TZP controlling morning-specific growth through these genes. In addition, two homeobox-leucine zipper genes induced in TZP-OX (HAT4 and HAT52) are upregulated under low light conditions and in response to auxin (SI FIG. 12C). Mutations in these genes lead to growth defects (Sawa, S. et al., Plant J, 32:1011-1022 (2002); Sessa, G. et al., Genes Dev, 19:2811-2815 (2005)). Together, these results support the notion that TZP plays a broad role in the regulation of phytohormone-dependent gene transcription (Michael, T. P. et al., PLoS Biology, (in press) (2008)).

A similar number of genes were found to cycle across all three genotypes as reported for this condition previously [(Michael, T. P. et al., PLoS Genet, 4:e14 (2008)); ˜7,700 genes], and there was no significant difference between the number of genes cycling in rHIF lines and TZP-OX, although there were genes specific to each genotype. However, the peak transcript abundance of 362 genes was shifted by six hours or more in TZP-OX compared to rHIF138-8 (only genes that cycled in both genotypes were considered). Phytohormone-related (SIR1, ETO1, GH3.3, RGA1, ABF4), homeobox-leucine zipper (HAT2, HAT3), chromatin remodeling (HD2B, HDT4, SUVH9, CHR4, TAF1), leaf polarity (KAN3, AS1) and ribosomal genes were misphased in TZP-OX. PHYTOCHROME D (PHYD) was also phased eight hours earlier in TZP-OX (SI FIG. 12L). The first phyD mutant was originally identified as a natural allele, and subsequently was shown to affect shade-avoidance associated growth and flowering (Aukerman, M. J. et al., Plant Cell, 9:1317-1326 (1997); Devlin, P. F. et al., Plant Physiol, 119:909-915 (1999)).

Consistent with TZP controlling blue light dependent growth, LONG HYPOCOTYL IN FARRED 1 (HFR1) is overexpressed at dawn more than 100 fold with the same pattern as IRX1 (SI FIG. 12I). HFR1 encodes a bHLH transcription factor that is required for both phytochrome A-mediated far-red and cryptochrome 1-mediated blue light signaling (Fairchild, C. D. et al., Genes Dev, 14:2377-2391 (2000)). HFR1 expression is high under low light conditions such as shade and continuous dark conditions (Sessa, G. et al., Genes Dev, 19:2811-2815 (2005)), and is elevated in circadian and light signaling mutants, much like in TZP-OX [SI FIG. 12I; (Michael, T. P. et al., PLoS Biology, (in press) (2008))]. Recently it has been shown that two other bHLH transcription factor genes related to HFR1—PIF4 and PIF5—control morning-specific hypocotyl growth. Disruption of the circadian clock gene CCA1 results in the overexpression of PIF4 and PIF5 leading to uncontrolled elongation (Nozue, K. et al., Nature, 448:358-361 (2007)). However, PIF4 and PIF5 are expressed at control or slightly lower levels in TZP-OX (SI FIGS. 12G-H). These results support TZP acting in parallel with (or downstream of) PIF4 and PIF5 in growth control.

Conclusions: Despite extensive forward genetics screens in A. thaliana, natural variation has recently made important contributions to the identification of genes not previously known to impact several different traits [e.g., (Bentsink, L. et al., Proc Natl Acad Sci USA, 103:17042-17047 (2006); Mouchel, C. F. et al., Genes Dev, 18:700-714 (2004); Macquet, A. et al., Plant Cell, 19:3990-4006 (2007); Baxter, I. et al., PLoS Genetics, 4:e100000438-41 (2008))]. Apart from being able to exploit allelic variation (in multiple genetic backgrounds) that cannot be generated by conventional mutagenesis, the success of these studies has often been due to the use of quantitative phenotyping, as opposed to the qualitative gauges employed in typical mutant screens. We have demonstrated here the power of QTL analysis to reveal a new component of the hypocotyl growth pathway in A. thaliana, TZP, a unique, tandem zinc knuckle/PLUS3 domain protein encoded by a single copy gene in the vascular plant lineage. TZP provides a direct link between light signaling and the pathways that control growth in an environmentally independent fashion.

### Materials and Methods

A detailed and referenced version of this section is available online (SI Text).

Plant material and phenotyping. The Core-Population of 164 RILs from the Bay-0×Shandara set (http://dbsgap.versailles.inra.fr/vnat/) was phenotyped in four different light and temperature environments to map QTL affecting hypocotyl elongation. Complete phenotypic data from RILs is available in table S14 from our website data at www.inra.fr/vast/Files/Loudet_PNAS_SITables.xls. HIF350 was developed from an F7 line (RIL350) that still segregated for a single and limited genomic region around LIGHT5 locus. Plants still heterozygous for the QTL region were screened with adequate markers to isolate recombinants (rHIF) used in the fine-mapping process. Advanced rHIF crosses were generated from two different rHIFs recombined immediately to the north or immediately to the south of the LIGHT5 interval giving rise to lines arHIF47. Distinct Bay-0 lines from the stock center were used to find variants at LIGHT5. rHIF138-8[Bay] was complemented by over-expressing each of the three positional candidate genes cloned from rHIF138-13[Sha].

QTL mapping. Analyses used hypocotyl length mean values of an average of 16 seedlings (from two distinct experiments) per genotype per environment. QTL analyses were performed using QTL Cartographer, with classical parameters for interval mapping and composite interval mapping.

Microarray analysis. Microarray experiments were carried out per Affymetrix protocols (ATH1 GeneChip), on seven-day-old tissue harvested under either continuous blue at subjective dawn, or every four hours (starting at dawn) under 12 hrs white light/12 hrs dark cycles over one day (six time points). Hybridization intensities from all microarrays were normalized together using gcRMA implemented in the R statistical package. The blue dataset was then separated and differentially expressed genes were identified using linear modeling with the limma bioconductor package in R.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### SEQUENCE LISTING

SEQ ID NO: 1
- Arabidopsis LIGHT5 coding sequence
ATGGGAGATGGAGATGAGCAAAGTAAAGAGTTAGGAGGAGTTTCGTCTTC
GAGTCGTCGTTGTTCTTCCGGTACTGCTGGTGCTGCAAATGCAGAAGCAA
GAATGAAGTTTGCTGCTGTTGATGCTATAACAGAATTAGTGTGGTCTCCT
AGCAACGGTCTAAGCTTAAGATGTGCAGATATCAGTTTCACGGGAAAGGC
AAAATTACTCTCTCCTAACTTCTTTGATATAGGACTAACTAACATGGCGA
TTCACTCAAATAGCACAAGTATTGAAGACCAAGAAGATCACGTAGATGTC
GAATTGAGAAACCGAGATCAAGTGAACCAAGCAATGATTGGTGGTTCTGT
TGAGGATATGAAACCTGAAATGGTTGAGGATAAGGTTGAAACCAATGATG
ATATAAAGAATGAAGAAGCAGGATGTTCCAAAAGATCTTCAGATAGTCCG
AAAGCTATGGAAGGAGAAACCAGAGACTTGTTGGTTAATGAACAGTTGAG
GATGGAAAGTGCTGGATCTCAAGAAGAAGGGGATAAGGCGCATAATAGAG
TTGATCGTTTAGAATCAATGGATGAGAACAACCTCGCAACTCTTGCTGTT
GTAGCATGTGAAGGTAAAGGGGACTATTTGCCGGAGGGTGAGGCTGGACC
TAGTGGTTCCTACAGGCGTCGTGAGAAAGCAAAAGGGAAAGAGAAAGCTT
TATCTGATGAAAACTTTGGTGGTGATGGTGAAGATGAGGATGAGGAGAGT
TTTGGTAGTGTAGAAAGTTGCAATAGTGCGGGTTTGCTCTCAAGGGGAAA
GAAAAGACCAGGCTTTGAGGAGCAGCTGATTTTTGGAAGCAAACGGCTCA
AAACGTTAAATCAAGAATGTTTGGGGTCAACTTCAAAGCTCAAGCAAGAT
AGTTCCTTCATGAATTGGATATCAAATATGACTAAAGGAATCTGGAAAGG
TAATGAAGAAGACAATTCTCCTTTTGTAGCTCTTACCACCACCTCAAATG
CTAATGGTCATGGCCAAGTCAATGCCATCGTTGACCAACAACAATTGAGT
CCATGCTGTGTGAAGGAAAATAGTGGATGCAGAAACACCGGTTTCCAGTC
TTTTTTCCAGTCTATATATTGTCCGAAGAAACAAAGTCAAGATGTCGTCG
ACATGGATTTCCCGAATGATGTTAATGCTGCTCCTTTGCAGGAACTTCCC
TGGATTCCTGAACATTGCGACATTTCCAAAGGGGATGACTTGTCTTCATC
TGGTAATGAGATTGGTCCAGTGGCTGAACCCAACATTTCATCAGGAAAAG
TTGTATTTAATCAGACAAGCAAAACACAGTCTTCAGAGAACAAACGTGAG
GACAAAGAGCCAAACATCTCGTTGATGTCTCTTAGTAAGTCTAAGCCAAA
TGAAGAGCCAAAAACATGCGGTGAAGCTGATGGAAAGGTTAGTCCATGTT
TAACCAACAGAAACTCTGGTCTTAAAAGTTTGTGGATTAGTCGGTTTTCT
TCTAAAGGTTCGTTCCCTCAGAAAAAGGCAAGTGAAACGGCTAAAGAGGC
CAATGCTTCAGCCTCAGACGCAGCTAAAACCCGTGATTCACGGAAAATGC
TGGCAGACAAGAATGTTATAAGACCCAGTATCAGCTCGGTAGATGGGCCC
GATAAACCGGACACTGTCCTTCCTATAGTTTCGTCAATGAGAATAGAGTC
TTCAGAAGCAATGGCTTCTTTGTTTGCTAGAAGATTAGAAGCAATGAAAA
GCATCATGCCCTCAGGTTCTTTAGCTGAGAATGCAGAGGAGGAACAGAGG
GATCTAATTTGTTTCTACTGCGGTAAAAAGGGTCATTGTTTACGGGATTG
TTTGGAAGTAACTGATACTGAGCTCAGAGATCTAGTACAGAACATAAGTG
TGCGGAATGGAAGAGAAGAAGCGTCAAGTCTATGCATTAGATGTTTTCAG
CTTAGTCACTGGGCTGCAACATGCCCGAATGCTCCCCTGTATGGTTCAGG
AGCTGAAGGTAGAGCTATGAAGAACGCTTTAGCTTCTACCTCTGGCATGA
AGCTGCCCATAAGTGGTTTCACAGACGTACCAAGGGCGGTCTTTGATGCT
GTTCAAGTGCTTCGCCTGAGCCGCACAGACGTTCTCAAATGGATAAACAC
CAAGAAGTCTGTATCGGGTCTCGAAGGGTTCTTCTTGCGGTTAAGGCTCG
GGAAATGGGAAGAAGGGCTTGGAGGAACAGGGTATTATGTAGCTCGCATA
GATGGGGACACAGAGGGACAAAGCTCAAGGAGACATTCAGAGAAAAGCTT
AATTTCAGTTAAGGTTAAAGGTGTGACTTGCCTTGTTGAGTCTCAGTTTA
TCTCAAACCAAGACTTTCTTGAGGAGGAGTTAAAGGCCTGGTGGCAGAGC
GCGGGGAAAAGTGCCAGAACAAGCGGCTACGACGGCATCCCGTCGGCGGA
GGAACTTAGTCGGAAAATTCAGCAGAGAAAAATGTTAGGCTTTTAG
SEQ ID NO: 2
- Arabidopsis LIGHT5 amino acid sequence
MGDGDEQSKELGGVSSSSRRCSSGTAGAANAEARMKFAAVDAITELVWSP
SNGLSLRCADISFTGKAKLLSPNFFDIGLTNMAIHSNSTSIEDQEDHVDV
ELRNRDQVNQAMIGGSVEDMKPEMVEDKVETNDDIKNEEAGCSKRSSDSP
KAMEGETRDLLVNEQLRMESAGSQEEGDKAHNRVDRLESMDENNLATLAV
VACEGKGDYLPEGEAGPSGSYRRREKAKGKEKALSDENFGGDGEDEDEES
FGSVESCNSAGLLSRGKKRPGFEEQLIFGSKRLKTLNQECLGSTSKLKQD
SSFMNWISNMTKGIWKGNEEDNSPFVALTTTSNANGHGQVNAIVDQQQLS
PCCVKENSGCRNTGFQSFFQSIYCPKKQSQDVVDMDFPNDVNAAPLQELP
WIPEHCDISKGDDLSSSGNEIGPVAEPNISSGKVVFNQTSKTQSSENKRE
DKEPNISLMSLSKSKPNEEPKTCGEADGKVSPCLTNRNSGLKSLWISRFS
SKGSFPQKKASETAKEANASASDAAKTRDSRKMLADKNVIRPSISSVDGP
DKPDTVLPIVSSMRIESSEAMASLFARRLEAMKSIMPSGSLAENAEEEQR
DLICFYCGKKGHCLRDCLEVTDTELRDLVQNISVRNGREEASSLCIRCFQ
LSHWAATCPNAPLYGSGAEGRAMKNALASTSGMKLPISGFTDVPRAVFDA
VQVLRLSRTDVLKWINTKKSVSGLEGFFLRLRLGKWEEGLGGTGYYVARI
DGDTEGQSSRRHSEKSLISVKVKGVTCLVESQFISNQDFLEEELKAWWQS
AGKSARTSGYDGIPSAEELSRKIQQRKMLGF*
SEQ ID NO: 3
Arabidopsis LIGHT5 genomic sequence
GGCGCTGTGTCGTCTTCGTCTTCTTCTTCCTTCTTCTTCTTCATCTCTCT
CTTTCGTTTTCTCAGTAAGTGAAGGATTTGCAGAAGTAGATGATTGAATA
TCCATGGAGGTAATTTATGGTTTCTAATTTGGTTTTAATTCTTTGAAAGT
TTGATTTTTTTATGTTATGAGATTTCTGGGTTTTTGATTCTCAAGTTTTT
ATGGATTTTAGGGTTTTCAGTAATTGCAAAACTTTATTCTGAAGATTGAG
TGATTGCTTGAGATATAGGTTATCAATTTCCTTTATCCCTAATATTCCAA
ATCTCGAAATTTCAAATTACTTCTCTAAGTTGTTCTATGAGTTTATGATC
TTAGAAAATAAATCTAGTGTTTTGATTACTCTGTTTTGCATATTTTTGAT
GGGTTTGTGTTACTTGTTTTGTAAGCTAAGTGATATTCATTTTTGTGGTT
GAGGTCAATAGTGTATGGATTCTGGTTCTTTATGTCTCAGGGTGATTGGT
TTCAGGAGGTATGGGAGATGGAGATGAGCAAAGTAAAGAGTTAGGAGGAG
TTTCGTCTTCGAGTCGTCGTTGTTCTTCCGGTACTGCTGGTGCTGCAAAT
GCAGAAGCAAGAATGAAGTTTGCTGCTGTTGATGCTATAACAGAATTAGT
GTGGTCTCCTAGCAACGGTCTAAGCTTAAGATGTGCAGATATCAGTTTCA
CGGGAAAGGCAAAATTACTCTCTCCTAACTTCTTTGATATAGGACTAACT
AACATGGCGATTCACTCAAATAGCACAAGTATTGAAGACCAAGAAGATCA
CGTAGATGTCGAATTGAGAAACCGAGATCAAGTGAACCAAGCAAGTAATG
AAATGAATAGACCATTTTCAGGTTGCTATATGATAAGTTATCTTTTTTGA
TAATGAGAGTTCTTGTGTGTTTGCAGTGATTGGTGGTTCTGTTGAGGATA
TGAAACCTGAAATGGTTGAGGATAAGGTTGAAACCAATGATGATATAAAG
AATGAAGAAGCAGGATGTTCCAAAAGATCTTCAGGTAAAATGGTTATATC
AAGTTAATAATGTTTGCGTCTCTTCCATTTATGACAACTATGGATTTTGA
GTTGGTTTCTTCGTCTTTTGTGCTTGTTTTGGTTATCGAGTAGATAGTCC
GAAAGCTATGGAAGGAGAAACCAGAGACTTGTTGGTTAATGAACAGTTGA
GGATGGAAAGTGCTGGATCTCAAGAAGAAGGGGATAAGGCGCATAATAGA
GTTGATCGTTTAGAATCAATGGATGAGAACAACCTCGCAACTCTTGCTGT
TGTAGCATGTGAAGGTAAAGGGGACTATTTGCCGGAGGGTGAGGCTGGAC
CTAGTGGTTCCTACAGGCGTCGTGAGAAAGCAAAAGGGAAAGAGAAAGCT
TTATCTGATGAAAACTTTGGTGGTGATGGTGAAGATGAGGATGAGGAGAG
TTTTGGTAGTGTAGAAAGTTGCAATAGTGCGGGTTTGCTCTCAAGGGGAA
AGAAAAGACCAGGCTTTGAGGAGCAGCTGATTTTTGGAAGCAAACGGCTC
AAAACGTTAAATCAAGAATGTTTGGGGTCAACTTCAAAGCTCAAGCAAGA
TAGTTCCTTCATGAATTGGATATCAAATATGACTAAAGGAATCTGGAAAG
GTAATGAAGAAGACAATTCTCCTTTTGTAGCTCTTACCACCACCTCAAAT
GCTAATGGTCATGGCCAAGTCAATGCCATCGTTGACCAACAACAATTGAG
TCCATGCTGTGTGAAGGAAAATAGTGGATGCAGAAACACCGGTTTCCAGT
CTTTTTTCCAGTCTATATATTGTCCGAAGAAACAAAGTCAAGATGTCGTC
GACATGGATTTCCCGAATGATGTTAATGCTGCTCCTTTGCAGGAACTTCC
CTGGATTCCTGAACATTGCGACATTTCCAAAGGGGATGACTTGTCTTCAT
CTGGTAATGAGATTGGTCCAGTGGCTGAACCCAACATTTCATCAGGAAAA
GTTGTATTTAATCAGACAAGCAAAACACAGTCTTCAGAGAACAAACGTGA
GGACAAAGAGCCAAACATCTCGTTGATGTCTCTTAGTAAGTCTAAGCCAA
ATGAAGAGCCAAAAACATGCGGTGAAGCTGATGGAAAGGTTAGTCCATGT
TTAACCAACAGAAACTCTGGTCTTAAAAGTTTGTGGATTAGTCGGTTTTC
TTCTAAAGGTTCGTTCCCTCAGAAAAAGGCAAGTGAAACGGCTAAAGAGG
CCAATGCTTCAGCCTCAGACGCAGCTAAAACCCGTGATTCACGGAAAATG
CTGGCAGACAAGAATGTTATAAGACCCAGTATCAGCTCGGTAGATGGGCC
CGATAAACCGGACACTGTCCTTCCTATAGTTTCGTCAATGAGAATAGAGT
CTTCAGAAGCAATGGCTTCTTTGTTTGCTAGAAGATTAGAAGCAATGAAA
AGCATCATGCCCTCAGGTTCTTTAGCTGAGAATGCAGAGGAGGAACAGAG
GGATCTAATTTGTTTCTACTGCGGTAAAAAGGGTCATTGTTTACGGGATT
GTTTGGAAGTAACTGATACTGAGCTCAGAGATCTAGTACAGAACATAAGT
GTGCGGAATGGAAGAGAAGAAGCGTCAAGTCTATGCATTAGATGTTTTCA
GCTTAGTCACTGGGCTGCAACATGCCCGAATGCTCCCCTGTATGGTTCAG
GAGCTGAAGGTAGAGCTATGAAGAACGCTTTAGCTTCTACCTCTGGCATG
AAGCTGCCCATAAGTGGTTTCACAGACGTACCAAGGGCGGTCTTTGATGC
TGTTCAAGTGCTTCGCCTGAGCCGCACAGACGTTCTCAAGTAAGATCTAA
TTCACACCTAAAAGACTGTTTCCCTTGTCACATTATGAGGCTTTAACATC
TTTTGACATTTCATTTCAGATGGATAAACACCAAGAAGTCTGTATCGGGT
CTCGAAGGGTTCTTCTTGCGGTTAAGGCTCGGGAAATGGGAAGAAGGGCT
TGGAGGAACAGGGTATTATGTAGCTCGCATAGATGGTTGGTACACTTTCA
TATAAATCAATACTCAAACTCGCGTATTTCTCGAATATTTGCAACTGAAA
TATGTTTGTTTTAACCAGGGGACACAGAGGGACAAAGCTCAAGGAGACAT
TCAGAGAAAAGCTTAATTTCAGTTAAGGTTAAAGGTGTGACTTGCCTTGT
TGAGTCTCAGTTTATCTCAAACCAAGACTTTCTTGAGGTATATAATAAAA
TTTGACCAATTATGTCAAAAGTTTTGAAATTTGCTAATTTGCTAAGTTTT
CATCTGAATATTTTGCATTAATTTATCTGAGCAGGAGGAGTTAAAGGCCT
GGTGGCAGAGCGCGGGGAAAAGTGCCAGAACAAGCGGCTACGACGGCATC
CCGTCGGCGGAGGAACTTAGTCGGAAAATTCAGCAGAGAAAAATGTTAGG
CTTTTAGGACATTTGTTCAAGTAGATAAATGTTTTTAAGTTATAGGTTTT
ACGATTACCCGTGTATTTTTTCGGTTGAAAAGGAGAGTGTAAATTTCTTA
GTTCAGAGGGCATCGAATGTCTCAAACCATACAACAATCTAAAACTGGAT
GTCCAATCTATTTAGATTTGCGAGTTTTATTCGGATCTTTATGTCTCGAT
GACTATTTATTGTGATTGTTAAGCTTGTATAGTCTATGTGTGAAGGATCA
AAGTATCATGGAAGAACAAAGTACATTGATATCAAGAGAGATATTGTGGT
GTCTTGCTCATTTTTC
SEQ ID NO: 4
>GmTZPa_soybean
MMLPCDSRIHMAINKGKEKSLSDGDANVILSREENDSHSSVESCNSAGFF
STGKKRRNFQQQLIIGSKRVKKQIEESSGFKSYVKQDSSFMNWISNMVKG
LQQSIQNDSNTLALTLTNPDHHNLLPDEKLFTCNMNQDPEPKNTGFKSFF
QSIYCPSLKNGGTRMSHQEGKSSDDLEPGNMEHGIDATPITYCAENNSLS
KLRLQSNKFEVSIGGNDAGPSSQPKVKPLNFFNCQESSKNNPVETKNYSI
LGHSKDKEEVASHSSSTKQNTDDNDNIDSNALPDRKEEENICHRRDNLGS
LWITRFSPKFTAPLREQPANDTEASTDLKEDKGNNDHKSMYMFKPLSSSP
GLRNLEPMASMFARRFSAIKHIIPTNATDTTTQVNMLCLFCGTKGHQLSD
CSAIAENKLEDLQKNIDSYGGLEEHSCLCIKCFQPNHWAISCPTSISTRK
HELKANALVNDCGKQKHLIPSNEESARLLTDEDDRVLSGGSINDETDQRT
GQNINLKLKSNEIITHKVGCNASFQKYCGSSLEENKFRENPISSPSKLTE
RQISHVPKKIFDAVKKLQLSRTDILKCINTHGSISQLDGFFLRLRLGKWE
EGLGGTGYHVAYINETQSQRQCPEQNTRKCLSVKVGSIKCMVESQYISNH
DFLEEEITEWWSNTSEAGAEIPSEEYLIEKFKKKEMLGL*
SEQ ID NO: 5
>GmTZPb_soybean
MLPCDKILPVLHSPCHSRIHMAINKGKEKSLSDGDANVMLSREENDSHSS
VESCNSTGFFSTGKKRRNFQQQLIIGSKRVKKQIEESSGPKSYVKQDNSF
MNWISNMVKGLSPSIQNDSNTLALTLANPDHHNLQPDEKLIACNMNQDPE
PKNTGFKSIFQSICCPSLKNVGTRMSHQEGKSSQDLVPGNMEHGIDATPI
TCWAENNSLSKLCLQSNKFEVSTGGNDAGLSSQPKIKPLNFFNCHESSKN
NPVETKNYSILGHSKDKEEVASHSSSTKQNTDNNDNIDSNVLCDRKEEEN
ICHRRDNLGSLWITRFSPKFTAPLREQPANDTEVSTDLKEDKGNNDHKSM
YLFKPLSSSPGFRNLEPTASMFGRRFGAIKQIIPTNATDTTTQVNMLCFF
CGTRGHQLSDCLAIAENKLEDLQKNIDSYGGLEEHHCLCIKCFQPNHWAI
SCPTSISTRKHELKANALVNDCGKHLISSNEGSARLLTDEDDRVLSGGSI
NDETDQRAGQNINLKWKSNEIITHKVGCNASFKKYRGLSSEENKFRENPT
LSPSKLAERQISQVPKEIFEAVKKLQLSRTDILKWINTRGSISQLDGFFL
RLRLGKWEEGPGGTGYHVAYINETQSQRQCSEQNTRKSLSVKVGSIKCMV
ESQYISNHDFLEEEIMEWWSNTSQAGAEISSEEYIIEKFKKKEMLG
SEQ ID NO: 6
>OSTZP_rice
MDVMNEGKETSDNFCVDKLEKEDEVGSCPTRYCNDTSHSLGSASRKEVMP
IIAEKQAFCATTVHDERSWAANAWRARLVKAISQKDSVLPKNADNIHSTS
AFGSIGNTENMPGKLTSMLGNRNDSSQDQAMQEKHKDGLIVARCESVSAV
NPVARCELASGVNPLARHESTSGCNPRKLEKGKEKLIYDMSNCVSNTNEG
DDSNESIESCPSTKAPKRKHGQFSAAEMTSGNKRCRREDNESSCSGLFHK
NDSSFFNWMSTLTNGVKVFDETTAVPLNQKFSAATGEEFPTNPVPLQNNC
GVPLQSVGFNSLFQSLYSQNVMITSRNTCHQSESSYTANRLTLGFKSSKP
VSMGRETLNVATETLAAGRIQMDSYGDRGAFQNQMGIFPLRAERNQNGFH
GSSSNAASGHKDDFSESLWVSRLLPKTPMKVMDTTRCDEETDFCSANPKG
LGDSSSPQDFNVEKELNNSQYFTSKGSDNETTSSKCAAPQDENKPSETMA
SIFAKRLDALRHANTSAVHVAITCDHGTPKGRNHKTSSFVVSYNSHDEQE
SGQKTHKSSGGEGRIVLWTGDKGKEQLSPGNDKELGEKVLSKHENQNCEG
SSDGKVVPPKCNLETNTYIEEIDRKRLQNKEGAPNSMENQPDNKQMVPYG
IVPNDVYDEASVVFGALQRLRLSRSDIIRWMRSPVMHTTLDGFFLRLRFG
KWEEALGGTGYHVARINGVLDKNRLSVTIRNSTCQVDSRFVSNHDFHQDE
LKAWWSAAMKSGWKLPSQEELNTKLRERELLRF
SEQ ID NO: 7
>PtTZP_poplar
MDTNDKNIEPVIDLGFSLGYSNQCIQRRLKNDSGAGANAASSVDMTFVAT
NALSELVWSPKKGLSLKCADGTFSNQKPSLLRGAGPSDMVSGSNADKAIG
KKVFMTPPEESDVRSEVAGRDNPTKFVTSDTGLFPLLSESMHKVKIGNYE
FLAATDDHKEEMKTAVGLPFLQKMEDARNNKAEDIYDPINLQVDEISRTW
ETKFPSLSDETKLDVAQNGPTSKEPNVRIGGVGDASHTLQTEIVSASQVC
SVEECESYDTNMQKAPLGREHFESPSCMEKERENNMGTGPYICPLEKLES
TAENDFKTPHSENVCAVATEIVGSQNAKEVRSSSQQDDEILPKDNDCAIK
QSPTYSRTRRYQMKGKAKALSDGNLNERMLDMDDDSHESVESCNSVGLFS
TGKRQRNFDPHSYVGSKSIKTKIQESPGSSSFVKHDGSFMNWISNMMKGF
LKSNEDEAPSLALTLANHKHGHEDRDKNLISCNRNQDQGCKTMGFHSLFQ
SLYCPKTKAQETVALNANTQTEGSKELGLDNKICDSNATPIPCRMVTDNV
YKRFLQPNEKLNESTSGNGTASPALTKLLSTNIASGQEISGSNSAEKKNS
CNMATDKEKNGTSSNSSRGKRKMNDAEQPSEGKATNTSGYRSDPLTSLWI
TRLSPKTSGPLSNRDLCHRRTGEALDGFTDFIRLKAQWQNHPSSYQDKNI
VGAREEEHFTEDPVCMHNCANSTEVSFSINKVNGHHDEKSMCKMNSTLPF
SRFRNSEAMASVFARRLDALMHIMPSYGTDDSSHGNLTCFFCGIKCHHVR
DCPEIIDSELADILRNANSFNGANEFPCVCIRCFQSNHWAVACPSASSRT
RHQAEYGASLVHESSPCKILLNPRNEDDAKQSDGKDSQLQAADAPTVRNG
KLHEASASGKINMNMKPFERDTASSSGEKKLKENQVMPLSNFINSQIADV
PKGIFDAVKRLRLSRTIILK
SEQ ID NO: 8
>CpTZP_papaya
MNVDKDNLEPHTDLGLTVNYSNHCIQRRLNNHLGAGANAGSSRGMTFVTT
DPLSELVWSPQKGPSLKCADCSFSDKKSSLLWDAGPSNVVFSSAPPMISA
VRCSNDKLMNENNNMIKSHMGFYVKSDVGDRHTSARCPTNNAAIKPVSGP
SPEQKTGTGGHMEETSTILQLSVQHANKNDDLSRPKGEVTCDLNTIQADE
AAEAIEDNLKNFLDVLRPNVAQTEPLFENPAGGYCDANSENQTSKIEINL
MSDIHAKNKTEACDDALKSQTVPPKRREEPASFTEGENDRKIKSTDSSNI
RCMEKLESTAENDLQIHIGENGCDAASKSVATEFAHEGKRCSQQEKARFR
EKLASGKYSAKNSGIQKYERKGKEKALSDGNLSMMSKEADDSYESVESCN
GTGLFSTGKRRWNFEQQMIAGSKRAKMQIRETLESTSFVKQDSSFVNWIG
NMMKGFLKLKEDEAPSVSLTLAYPTNGGPDHDTIATTRNKNPGCRTVGFQ
SIFQSIYCPQRRIQDVTTSNDKCHKEVELPNDIFNPNPVNCHGNFTKQFT
ISDERNTESTFGNGVLLETQPKISSVNFAASQRTRIPTLQRQMVVQFAQY
KGKEGTSSSSSLGKYKLNIAENIDSEPLSEGESGQNFGNGSDHLGSLWIA
RLAPKTSGFSLNLNQQNRSTDVDLEYSADCAKLPHSPQYHVSSPSEAKIV
EARQHSLDDQKIAIGNDLPKCAGETECKITCNSAKDHNDHKSTYNMNPIL
SSPIENSEAMASLFARRLDTLRHIMPSDLVENTASATVSCFFCGRKGHHL
RDCPEMTDEDLEDLLRTVNKYNGTEEFSSFCVRCFRLNHWAVTCPNASSK
GNRQRECGASLAGEFSPSSGKHNSRIEENPKFLNGNDSQFQVAEVHSLLD
RNDSGIRASLKLNSTEKLVASSSGKNKLRGNEIVPLSKYITRESSDVPKG
IFDAVRMLRLSRTDILKWMNSQMAGSHLDGCFLRLRVGKWEKGLGGTGYY
VACITEAQRQSSPQSSKNSIFVVVRGIRCLVESQYISNHDFLEDELNAWW
FTIEKNGGTIPMEEVLRLKDRHKYNLYIGVVCYVDKKF
SEQ ID NO: 9
>RcTZP_castorbean
MNVDDKNIEPSTNLSLALGYSNQCIQRNLSNDPGAGANAASTADITFVAT
DPLSELVWSPHKGLSLRCADGSFIDKKPSLLPGVGPTYMASGSSSDKPIS
NTGKLFDNEICIASLPACKLASEISGDNSTTFLTSNVGIMPLSGTGLDKT
ATGDQVVEMKNAVNYFLQKEDLRNDKAEDETKLDVAQNYRTFEEPIVRAT
DVNDDHELGMEIVLVSDFHTVKGREDYGIKIQNAACSGKENEEPPSVREK
ERKNKMVIGRPGIFSLDKLESTAENDLETPFGENSCSMRNKNLASESADR
VENNTQHELIPIEYALGYNQSPTSSRLQNIQRQGQSKALSDGDAKERMLN
EEDGSHESVESCNSTELFSTGKQRWNFDQQLIVGSKRVKRQIQDSPGSSS
LGKQDSSFVNWISNMMKGFLKSSEGEAPFLSSALSNPNYGHENPSQDVFT
CNRKEDPACDTRGFQSVFQSLYCRKTKGQETVTLNVNHQTEGSKECDQDN
KICDLNAAPIACRMVTGNVYKRFLPSNEKHNEPTSGYHAGMTVHSRDISM
SFPVIPESNGSVSTENKNSCNLAIGKEKDGTDSNFSHGKHKTSSAGKIDP
ELPSEDKTAHGEGYKGDPLGSLWIARFSPKTSGAPFNHYPSNKSTGEAFN
CSADSMGLIPQVQNPLGSSSEHEIVEVRNKNFQEPLPIQNYSTANRAPFD
FYNVKGNIDNDSGNKLNPILSSARVKTSEAMASVSPRRLDAPKYITPSDD
ADNSDRASMTCFFCGIKGHDLRECSEVTDTELEDLLRNINIYGGIKELPC
VCIRCFQLNHWAVACPSTCPRVRSKAECHASSVSHAGPSKSQLHVINEDD
TKAKNVTGSGHAICYGNDYGMDKDMNSWKSNEAATSGKMKLNIRLFEKNI
SSTSREKELKENQIIPLYGFVNGLISDVPNGIFDAVRSLRLTRTNILKWM
NSSASLSIDGYFVRLRLGKWEEGLGGTGYYVARITGMKSKKSIAVNVGGI
QCVIESQFVSNHDFLEDELKAWWSATSKVGGKLPSEKELRLKVEEKNXXG
GGGGNKIVIENHIVQYQ
SEQ ID NO: 10
>BdTZP_brachypodium
MCPLSELVWSPDDGLSIKIAASSLSTRKASLRWNADTLNIVISSPQQSSG
RGKSGDNIDATIADAGEMPSQPRTRCDSSVRLSMASPNRIRNLDAQQSTS
VRSQEQDSKCCGGISVMNKGKEVSQSCFVYNADKGEVGSCPTRCCKDVSD
GSASRKGVIPSISEKQVYCATTVENERPWADNAWRARLVKAISQKDYVLP
NNAMNAQSASSFEKFGNAEKVAGKLAGFLGKENDNHQDQVMQENHHGNHQ
DRIVQENHNDSHQYHVMQENHMDEPVLGRCGSASGGNPVSRCESASGVNS
AARYESSSGVNPTKLEKGKEKVMHDQSNCVSNIKEGDDSNESMESCQSMK
AQKREHAQCSIAEMSSRTKRCRREFNESSCSGFLQRNGSSFFNWVSSLTN
GLTVFDKSTTDVSLDQKFSVSTVHEFAEQSGPLQNNSSVPVQSVGFNSLF
QSLYRHNVMITSTDTCHQSEKKCTEHEADRVALALNDSNSMLGKQIGTSR
KTLDVPTETLAADSLLMDFGGGRGNFQNQIGVFPLRAGRNLMMPNSSKSC
SRSLEEKQNEAHAGSLKASVGNKGGFRESLWVSRLLPKTSMKLTDATPCN
IKSDFCAVNPKGAADKLYCSSQQNFSVEKEFNNSQYYTSTGSDNGTTSSK
CPAIPPEENKQSETMASILAKRLDALRHAKTSAVRLGNSCDQTISKECNH
GKSPFVVSYSGHDVQEARHETQKSSSGDGKLVLWLGDKGKGQLCTGSDEE
VRVKFLSGGDRQHCGGSMAGKAAAPHDNLEANTSAEYVQRRGVKIKEVLS
NSMESLPDNKQIVPYGIMSSDEYDRSSAVFGALERLRLSRSDIIRWLTSP
VRHTTLDGFFLRLRFGKWEEALGGTGYHVARINGALDRNRLSVTIRNSTC
QVDSRFVSNHEFHEDELKAWWSAAMKGGWKLPSNEELSKKLRERELLHPQ
NRTGQHNDT
SEQ ID NO: 11
>SbTZP_sorghum
MSPLSELVWSPDEGLSIKIAASSLSTRKASLAWNADTLSILISSPQHSGS
GPGVKSGDTIYDNLEVSEKMPSQLRIRSDSSVRVTMDSPNRVTNVDALQS
TSIRSQEQDSSSASRKEVMPSISENQVCCATTVRNERSWATNAWRARLVK
AVCQKEPMLSMNTENPMLPSSLGISCDAVEVSGKLVGSQGNRNVQSLGSD
NNVISNAPEIPSHNNCQDPVLQESHKDEPVVARGESASGVNAVARCESVP
GVNSRKLVKDKEKVLYNDSNYGNNTMEGDDSNESIESCTSTKAPKRKHAQ
FCAATMPSSGNKRFRREDNESSCSGLLQKCGTSFFNWMSSLTNGLPMLDG
ATAAIPLDQKFSASTGEGSAAPSQPLQNNSSVPMQSVGFNSLFQSLYTHN
VMITSRDNCHQPESNYAGHVFNRLTLELNDSNSMLDKQIGMGRETLDSGC
KKNQCTAACSNDATQNKGGLRESLWVTRLLPKASVELMEATPCNVENAVN
PQAVGDKLCCPPLQNFNLERGSSDGVTSSKCPATPPEEPKQSETMASVFA
KRLDALRHANTSAVRLAIACDRGSPKLRNHKTNSFVVSYSSHDKVEAGHE
NHKSSSRNGRIVLWLGDKGKEQLCLSNKESRGNFISEHEHQHHGGNTAGK
SATRPNLELNTLAEDTDRSQVKLKGGVSDFMAGPSDNKQIVPYGTMPNDV
CDESSVVFGALHRLRLSRSDIIRWLRSPIMHTTLDGFFVRLRFGKWEEAL
GGTGYHVARLNGALDRSRLSVTIRNSTCQVDSRFVSNHDFHEDELKAWWS
AAMKSEWKLPSKEELSVKLRERELLRS
SEQ ID NO: 12
>VvTZP_grape
MYRHRTKGKGKALSDGDRSGRKSNKEDDSDESVESCNSAALFSTGKKRWG
YEQQLITGSKRIRKQINGSPGSTSFVRQDSSFMSWISNMMKGLSKSNQDE
TPSLALTLARPNHDNYDQKLVTCNKNQDPGCRNIGFQSIFQSLYCPTTKV
QESRTLNADNQTGEGSKEFCLANKLCDFNQSTFGNRAGPSTQPKVLSAKF
AVSQENYKTSSVENRSASNPVSSSSSLGKRKANSAENNDSDPPSEGKTIH
NFGYKSDLLGSLWVTRFSPKTSSPTCKVDHCNQNTGGATELSTDCMGLIP
YSQNRTEVSFGFKKNNAHNNQNSIYKLNPISPSQRFKSSEAMASLFARRL
DALKNIITLNQTDTEARATPTCFFCGIRGHSIHDCSEIKETELEDLLRNN
NLYPGAEEPPCFCIRCFQLNHWAVACPSVLKRQNQSECGASLVNRCSSES
QIIPLCNFVNPQISDVPKGIFDAIKRLRLSRGDILKWMNSVFPFSHLNGF
FLRLRLGKWEEGLGGTGYYVACISGAQKERPSQSSKNPIAVNIGGVKCLV
QSQYISNHDFLEDELMAWWGATTRAGGKIPSEEDLKVKLEERKKFGF
SEQ ID NO: 16
>Populus2TZP
MEKERDNYMETGPYICPLEKLESTAENDFKTPHSENVCDVATEIVGSQNA
KEVRSSSQQDDEILPKDNDC
AIKQSPTYSRTRRYQMKGKVKALSDGNLNERMLDMDDDSHEKNDFKTPHS
ENVCAVATEIVGSQNAKEVR
SSSQQDDEILPKDNDCAIKQSPTYSRTRRYQMKGKAKALSDGNLNERMLD
MDDDSHESVESCNSVGLFST
GKRQRNFDPHSYVGSKSIKTKIQESPGSSSFVKHDGSFMNWISNMMKGFL
KSNEDEAPSLALTLANHKHG
HEDRDKNLISCNRNQDQGCKTMGFHSLFQSLYCPKTKAQETVALNANTQT
EGSKELGLDNKICDSNATPI
TCPMVTDNVYKRFLQPNEKLNESTSGNGAASPALTKLLSTNIASSQEISG
SNSAEKKNSCNMATDKEKNG
TSSNSSPGKRKMNDAEQPSEGKATNTSGYRSDPLTSLWITRLSPKTSGPL
SNRDLCHRRTGEALDGFTDF
IRLKAQWQNHPSSYQDKNIVGAREEEHFTEDPVCMHNCANSTEVSFSINK
VNGHHDEKSMCKMNSTLPFS
RFRNSEAMASVFARRLDALMHIMPSYGTDDSSHGNLTCFFCGIKCHHVRD
CPEIIDSELADILRNANSFN
GANEFPCVCIRCFQSNHWAVACPSASSRTRHQAEYGASLVHESSPCKILL
NPRNEDDAKQSDGKDSQLQA
ADAPTVRNGKLHEASASGKINMNMKPFERDTASSSGEKKLKENQVMPLSN
FINSQIADVPKGIFDAVKRL
RLSRTIILK*
SEQ ID NO: 17
>Gm3TZP
MSAENEKIKPKTDIELFLNNANQCIWKKLNNDSGAGANAASRADMTLAAT
DPLSEIVWSPDKGLSLKCADSSFAHKNSSL
LRDVGTSCMVFAPPQNFTGGSSTTDKPLDDDFLKPIAVVCAKSDIAEADA
PTMPPTGDSGVKAKCKAYEEDDIGSVGNKE
KVNTAATAPNLPNEQNGNLTNNWEKITGDQANSGTDKVSGIEGNRISAIS
EFFFCCSGPTCDVSFSVHRHSFVNSNGEGQ
ADQGPFDHLLLQSDENKPSMDQNPSPGRHSDGSVNIGLEKKAVVTDDDLH
TAVEPIIEYRGSGAHETNLASSSKNPLEKL
EYSAENDLQTFNCEAACAGTSRVNVSETENKFQDTEMMLPCDKILPVLHS
PCHSRIHMAINKGKEKSLSDGDANVMLSRE
ENDSHSSVESCNSTGFFSTGKKRRNFQQQLIIGSKRVKKQIEESSGPKSY
VKQDNSFMNWISNMVKGLSPSIQNDSNTLA
LTLANPDHHNLQPDEKLIACNMNQDPEPKNTGFKSIFQSICCPSLKNVGT
RMSHQEGKSSQDLVPGNMEHGIDATPITCW
AENNSLSKLCLQSNKFEVSTGGNDAGLSSQPKIKPLNFFNCHESSKNNPV
ETKNYSILGHSKDKEEVASHSSSTKQNTDN
NDNIDSNVLCDRKEEENICHRRDNLGSLWITRESPKFTAPLREQPANDTE
VSTDLKEDKGNNDHKSMYLFKPLSSSPGFR
NLEPTASMFGRRFGAIKQIIPTNATDTTTQVNMLCFFCGTRGHQLSDCLA
IAENKLEDLQKNIDSYGGLEEHHCLCIKCF
QPNHWAISCPTSISTRKHELKANALVNDCGKHLISSNEGSARLLTDEDDR
VLSGGSINDETDQRAGQNINLKWKSNEIIT
HKVGCNASFKKYRGLSSEENKFRENPTLSPSKLAERQISQVPKEIFEAVK
KLQLSRTDILK*
SEQ ID NO: 18
>CpTZP2
MNVDKDNLEPHTDLGLTVNYSNHCIQRRLNNHLGAGANAGSSRGMTFVTT
DPLSELVWSPQKGPSLKCADCSFSDKKSSL
LWDAGPSNVVFSSAPPMISAVRCSNDKLMNENNNMIKSHMGFYVKSDVGD
RHTSARCPTNNAAIKPVSGPSPEQKTGTGG
HMEETSTILQLSVQHANKNDDLSRPKGEVTCDLNTIQADEAAEAIEDNLK
NFLDVLRPNVAQTEPLFENPAGGYCDANSE
NQTSKIEINLMSDIHAKNKTEACDDALKSQTVPPKRREEPASFTEGENDR
KIKSTDSSNIRCMEKLESTAENDLQIHIGE
NGCDAASKSVATEFAHEGKRCSQQEKARFREKLASGKYSAKNSGIQKYER
KGKEKALSDGNLSMMSKEADDSYESVESCN
GTGLFSTGKRRWNFEQQMIAGSKRAKMQIRETLESTSFVKQDSSFVNWIG
NMMKGFLKLKEDEAPSVSLTLAYPTNGGPD
HDTIATTRNKNPGCRTVGFQSIFQSIYCPQRRIQDVTTSNDKCHKEVELP
NDIFNPNPVNCHGNFTKQFTISDERNTEST
FGNVREQEYQLFRDKWLCNLHNSKGKEGTSSSSSLGKYKLNIAENIDSEP
LSEGESGQNFGNGSDHLGSLWIARLAPKTS
GFSLNLNQQNRSTDVDLEYSADCAKLPHSPQYHVSSPSEAKIVEARQHSL
DDQKIAIGNDLPKCAGETECKITCNSAKDH
NDHKSTYNMNPILSSPIENSEAMASLFARRLDTLRHIMPSDLVENTASAT
VSCFFCGRKGHHLRDCPEMTDEDLEDLLRT
VNKYNGTEEFSSFCVRCFRLNHWAVTCPNASSKGNRQRECGASLAGEFSP
SSGKHNSRIEENPKFLNGNDSQFQVAEVHS
LLDRNDSGIRASLKLNSTEKLVASSSGKNKLRGNEIVPLSKYITRESSDV
PKGIFDAVRMLRLSRTDILKWMNSQMAGSH
LDGCFLRLRVGKWEKGLGGTGYYVACITEAQRQSSPQSSKNSIFVVVRGI
RCLVESQYISNHDFLEDELNAWWFTIEKNG
GTIPMEEVLRLKDRHKYNLYIGVVCYVDKKF*

1. A plant comprising a heterologous expression cassette comprising a promoter operably linked to a polynucleotide encoding a TZP polypeptide.

2. The plant of claim 1, wherein the promoter is heterologous to the polynucleotide.

3. The transgenic plant of claim 1, wherein the plant is characterized by increased size, speed of growth, elongation of organs or delay or extension of time of seed and/or fruit production compared to a plant lacking the heterologous expression cassette.

4. The transgenic plant of claim 1, wherein the promoter is a tissue-specific promoter.

5. The transgenic plant of claim 1, wherein the TZP polypeptide is at least 50% identical to SEQ ID NO: 2, SEQ ID NO:4, SEQ ID NO:5, SEQ ID NO:6, SEQ ID NO:7, SEQ ID NO:8, SEQ ID NO:10, SEQ ID NO:11, SEQ ID NO:12, SEQ ID NO:16, SEQ ID NO:17, or SEQ ID NO:18.

6. An isolated nucleic acid comprising a promoter operably linked to a polynucleotide encoding a TZP polypeptide wherein the promoter is heterologous to the polynucleotide.

7. The isolated nucleic acid of claim 6, wherein the TZP polypeptide is at least 50% identical to SEQ ID NO: 2, SEQ ID NO:4, SEQ ID NO:5, SEQ ID NO:6, SEQ ID NO:7, SEQ ID NO:8, SEQ ID NO:10, SEQ ID NO:11, SEQ ID NO:12, SEQ ID NO:16, SEQ ID NO:17, or SEQ ID NO:18.

8. A method for increasing size, speed of growth or biomass in a plant, the method comprising,
introducing an expression cassette into one or more plants, the expression cassette comprising a promoter operably linked to a polynucleotide encoding a TZP polypeptide, and
selecting a plant that has increased size, speed of growth, elongation of organs or delay or extension of time of seed and/or fruit production compared to a plant lacking the heterologous expression cassette.

9. The method of claim 8, wherein the TZP polypeptide is at least 50% identical to SEQ ID NO:2, SEQ ID NO:4, SEQ ID NO:5, SEQ ID NO:6, SEQ ID NO:7, SEQ ID NO:8, SEQ ID NO:10, SEQ ID NO:11, SEQ ID NO:12, SEQ ID NO:16, SEQ ID NO:17, or SEQ ID NO:18.

10. A method of producing processed plant material, the method comprising providing the plant of claim 1, or a part thereof; and
processing the plant or part thereof, thereby generating processed plant material.

11. A method of producing processed plant material, the method comprising providing the plant of claim 2, or a part thereof; and
processing the plant or part thereof, thereby generating processed plant material.

12. A method of producing processed plant material, the method comprising providing the plant of claim 3, or a part thereof; and
processing the plant or part thereof, thereby generating processed plant material.

13. A method of producing processed plant material, the method comprising providing the plant of claim 4, or a part thereof; and
processing the plant or part thereof, thereby generating processed plant material.
